(19) United States
(12) Patent Application Publication
(10) Pub. No.: US 2026/0306432 A1
(43) Pub. Date: Oct. 8, 2026

(54) NUTRITIONAL COMPOSITIONS, SUPPLEMENTS, AND FORTIFIERS

(71) Applicant: SOCIÉTÉ DES PRODUITS NESTLÉ S.A., Vevey (CH)

(72) Inventors: Cheong Kwet Choy (William) KWONG CHUNG, Lausanne (CH); Shreeram SATHYAVAGEESWARAN, Singapore (SG); Carla DELANNOY, Chexbres (CH); Guénolée Eliane Marie PRIOULT, Morristown, NJ (US); Maureen HUHMANN, Branchburg, NJ (US); Anupama DATTATREYA, Skillman, NJ (US); Priya KARKERA, Mumbai (IN); Shilpa WADWHA, Gurugram (IN)

(21) Appl. No.: 19/481,401

(22) PCT Filed: Apr. 26, 2024
(86) PCT No.: PCT/EP2024/061500

§ 371 Date: Nov. 4, 2025

Related U.S. Application Data
(60) Provisional application No. 63/464,322, filed on May 5, 2023.
(60) Provisional application No. 63/551,222, filed on Feb. 8, 2024.

(51) Int. Cl. A61K 31/198; A61K 9/00; A61K 31/07; A61K 31/355; A61K 31/375; A61K 31/4415; A61K 31/455; A61K 31/51; A61K 31/519; A61K 31/525; A61K 31/592; A61K 31/714; A61K 33/00; A61K 33/04; A61K 33/06; A61K 33/26; A61K 33/30; A61K 45/06; A61P 31/06

(52) U.S. Cl. CPC A61K 31/198; A61K 9/0056; A61K 9/0095; A61K 31/07; A61K 31/355; A61K 31/375; A61K 31/4415; A61K 31/455; A61K 31/51; A61K 31/519; A61K 31/525; A61K 31/592; A61K 31/714; A61K 33/00; A61K 33/04; A61K 33/06; A61K 33/26; A61K 33/30; A61K 45/06; A61P 31/06

(57) ABSTRACT

The present invention relates to a nutritional composition, supplement, or fortifier comprising L-arginine, L-citrulline, a vitamin mixture, and a mineral mixture. The present invention also relates to methods of enhancing immune responses, treating, preventing, and/or reducing the occurrence of infection, and reducing the occurrence or reoccurrence of one or more symptoms of infection, in particular in subjects with tuberculosis.

### FIELD OF THE INVENTION

The present invention relates to nutritional compositions, supplements, and fortifiers for dietary management of a subject with an infection and/or having an immunodeficiency, in particular subjects with tuberculosis. The present invention also relates to methods of enhancing immune responses, treating, preventing, and/or reducing the occurrence of an infection, or reducing the occurrence or reoccurrence of one or more symptoms of infection, in particular in subjects with tuberculosis.

### BACKGROUND TO THE INVENTION

Tuberculosis (TB) is an infectious disease caused by Mycobacterium tuberculosis and is a major cause of death world-wide. TB is highly prevalent among the low socioeconomic section of the population and marginalized sections of the community. In India, the National strategic plan (2017-2025) has a national goal of elimination of TB by 2025 (see e.g. Natarajan, A., et al., 2020. Indian Journal of Tuberculosis, 67(3), pp. 295-311).

Nitric oxide (NO) plays an important role in bacteriostatic and bactericidal processes as part of the host defense mechanisms against pulmonary infections. In particular, NO is a key anti-mycobacterial molecule and has a regulatory role in airway function and Mycobacterium tuberculosis proliferation and is therefore a potential target for anti-TB therapy (see e.g. Jamaati, H., et al., 2017. Frontiers in microbiology, 8, p. 2008).

L-arginine is the precursor for the endogenous synthesis of NO due to the activity of NO synthase, which releases L-citrulline as a by-product. It has been shown that L-arginine as an adjuvant to chemotherapy improves clinical outcome in active TB (see Schön, T., et al., 2003. European Respiratory Journal, 21(3), pp. 483-488). It has also been shown that L-arginine is useful as an adjunctive therapy in patients with active TB, in which the effects are likely mediated by the increased production of NO (see Farazi, A., et al., 2015. Tuberculosis research and treatment, p. 205016).

However, in a randomised, double-blind, placebo-controlled trial it was found that L-arginine supplementation did not affect TB subjects with pulmonary TB. In pulmonary TB, L-arginine may be degraded by arginases to ornithine (hence unavailable for conversion to NO), due to the predominance of immune responses favouring arginase production, such as macrophage type 2 (M2) responses. Increased arginase production is recognised in TB and arginase over-production has been hypothesised as a Mycobacterium tuberculosis-induced immune-evasion strategy (see Ralph, A. P., et al., 2013. PloS one, 8(8), p.e70032).

Mycobacterium tuberculosis (Mtb), has developed mechanisms to evade host defense responses and persist within host cells such as macrophages for prolonged periods of time. Mtb is also increasingly resistant to existing anti-TB drugs. There is therefore an urgent need to develop new therapeutics for TB and host directed therapies (HDTs) hold potential as effective therapeutics for TB.

Thus, there is a need for improved agents, combinations, or compositions for dietary management of TB. There is also a need for improved methods of treating, preventing, and/or reducing the risk of occurrence or reoccurrence of TB.

### SUMMARY OF THE INVENTION

The present inventors have developed improved nutritional compositions, supplements, and fortifiers comprising L-arginine, L-citrulline, a vitamin mixture, and a mineral mixture, for dietary management of a subject with an infection and/or having an immunodeficiency, in particular in subjects with TB.

The present inventors have also developed methods of treating, preventing, and/or reducing the risk of occurrence of TB in a subject, by administering L-citrulline. The present inventors have also developed methods of enhancing immune responses, treating, preventing, and/or reducing the occurrence of infection, or reducing the occurrence or reoccurrence of one or more symptoms of infection, in particular in subjects with TB, by administering a combination of L-arginine and L-citrulline.

In one aspect, the present invention provides L-citrulline or L-arginine to reduce intracellular load of Mtb in macrophages.

L-citrulline supplementation may increase plasma NO levels and reduce arginase activity. Moreover, supplemental L-citrulline may be more efficient than L-arginine in increasing systemic L-arginine availability. A combination of L-citrulline and L-arginine may act synergistically to enhance a subject's immune responses. Vitamins and minerals may further enhance the subject's immune responses.

In one aspect, the present invention provides nutritional composition, supplement, or fortifier comprising L-arginine, L-citrulline, a vitamin mixture, and a mineral mixture.

The nutritional composition, supplement, or fortifier may comprises the L-arginine and L-citrulline in any amounts suitable to enhance a subject's immune responses. Suitably, the L-arginine is present in an amount such that the nutritional composition, supplement, or fortifier provides L-arginine in a dosage of about 0.5 g/day or more. Suitably, the L-citrulline is present in an amount such that the nutritional composition, supplement, or fortifier provides L-citrulline in a dosage of about 0.5 g/day or more. Suitably, the L-arginine and L-citrulline are present in a weight ratio of from about 0.1:1 to about 10:1, from about 0.2:1 to about 5:1, from about 0.25:1 to about 4:1, from about 0.3:1 to about 3:1, from about 0.5:1 to about 2:1, or about 1:1.

The nutritional composition, supplement, or fortifier may comprise any vitamin mixture that is suitable to enhance a subject's immune responses. Suitably, the vitamin mixture comprises one or more of: vitamin A, vitamin D2, vitamin E, vitamin C, vitamin B1, vitamin B2, niacin, vitamin B6, folic acid, and vitamin B12. In some embodiments, the vitamin mixture comprises vitamin A, vitamin D2, vitamin E, vitamin C, vitamin B1, vitamin B2, niacin, vitamin B6, folic acid, and vitamin B12. In some embodiments, each of vitamin A, vitamin D2, vitamin E, vitamin C, vitamin B1, vitamin B2, niacin, vitamin B6, folic acid, and vitamin B12 is present in an amount such that the nutritional composition, supplement, or fortifier provides the vitamin in a dosage of about 50% RDA/day or more.

The nutritional composition, supplement, or fortifier may comprise any mineral mixture that is suitable to enhance a subject's immune responses. Suitably, the mineral mixture comprises one or more of: potassium, magnesium, iron, zinc, and selenium. In some embodiments, the mineral mixture comprises potassium, magnesium, iron, zinc, and selenium. In some embodiments, each of potassium, magnesium, iron, zinc, and selenium are present in an amount such that the nutritional composition, supplement, or fortifier provides the mineral in a dosage of about 15% RDA/day or more. In some embodiments, each of iron, zinc, and selenium are present in an amount such that the nutritional composition, supplement, or fortifier provides the mineral in a dosage of about 30% RDA/day or more.

The nutritional composition, supplement, or fortifier may further comprise any mixture that is suitable to enhance a subject's immune responses including antioxidants. Suitable antioxidants include one or more of: glutathione, coenzyme Q10, lipoic acid, flavonoids, phenols, polyphenols, and phytoestrogens. In some embodiments, the antioxidant is present in an amount that the nutritional composition, supplement, or fortifier provides a dosage of about 15% RDA/day or more. In some embodiments, the antioxidant is present in an amount that the nutritional composition, supplement, or fortifier provides a dosage of about 30% RDA/day or more. The recommended daily dose is equivalent to 8-11,000 antioxidant units a day.

The nutritional composition, supplement, or fortifier may further comprise any mixture that is suitable to enhance a subject's gastrointestinal health including fibers. Suitable fibers include one or more of: soluble fiber, insoluble fiber, inulin, or vegetable gums.

Soluble or insoluble fiber may be beneficial for alleviating symptoms of diarrhea or constipation and gastrointestinal discomfort.

Inulin and oligosaccharides may contribute to anti-inflammatory actions upon the bowel. They may increase absorption of calcium, magnesium, and iron.

Vegetable gum fiber such as guar gum and gum Arabic may further contribute to alleviating symptoms of gastrointestinal discomfort.

In some embodiments, the fiber is present in an amount that the nutritional composition, supplement, or fortifier provides a dosage of about 15% RDA/day or more. In some embodiments, the fiber is present in an amount that the nutritional composition, supplement, or fortifier provides a dosage of about 30% RDA/day or more. The recommended daily dosage is equivalent to 20 g to 30 g food fiber per day.

The nutritional composition, supplement, or fortifier may further comprise any other suitable components and may be provided in any suitable form. Suitably, the nutritional composition, supplement, or fortifier further comprises carbohydrate and/or protein. Suitably, the nutritional composition, supplement, or fortifier is in the form of a powder.

In one embodiment, the nutritional composition, supplement, or fortifier is a food for special dietary uses. The food for special dietary uses may be provided in the form of a powder for reconstitution with water or milk. The food for special dietary uses may be nutritionally complete.

In another embodiment the nutritional composition, supplement, or fortifier is a food for special dietary uses. The food for special dietary uses may be provided in the form of a liquid, gel or spoonable food. The food for special dietary uses may be nutritionally complete.

The food for special dietary uses may comprise the L-arginine, L-citrulline, vitamins, minerals, antioxidants and fibers and other components in any amounts suitable to enhance a subject's immune responses.

Suitably, the food for special dietary uses comprises L-arginine in an amount of about 1 g/serving or more. Suitably, the food for special dietary uses comprises L-citrulline in an amount of 1 g/serving or more. Suitably, the food for special dietary uses comprises: (a) L-arginine in an amount of about 1 g/serving or more; and (b) L-citrulline in an amount of about 1 g/serving or more.

Suitably, the food for special dietary uses comprises: (a) vitamin A in an amount of about 300 μgRE/serving or more; (b) vitamin D2 in an amount of about 5 μg/serving or more; (c) vitamin E in an amount of about 3 mgTE/serving or more; (d) vitamin C in an amount of about 25 mg/serving or more; (e) vitamin B1 in an amount of about 0.5 mg/serving or more; (f) vitamin B2 in an amount of about 0.7 mg/serving or more; (g) niacin in an amount of about 5 mg/serving or more; (h) vitamin B6 in an amount of about 0.6 mg/serving or more; (i) folic acid in an amount of about 60 μg/serving or more; and (j) vitamin B12 in an amount of about 0.7 μg/serving or more.

Suitably, the food for special dietary uses comprises: (a) potassium in an amount of about 250 mg/serving or more; (b) magnesium in an amount of about 30 mg/serving or more; (c) iron in an amount of about 4 mg/serving or more; (d) zinc in an amount of about 2 mg/serving or more; and (e) selenium in an amount of about 6 μg/serving or more.

Suitably, the food for special dietary uses comprises: (a) protein in an amount of from about 8 g/serving to about 14 g/serving, from about 10 g/serving to about 12 g/serving, or about 11 g/serving; (b) fat in an amount of from about 5 g/serving to about 10 g/serving, from about 6 g/serving to about 9 g/serving, or about 7.5 g/serving; and (c) carbohydrate in an amount of from about 20 g/serving to about 35 g/serving, from about 25 g/serving to about 30 g/serving, or about 27 g/serving.

Suitably, the food for special dietary uses comprises from about 100 to about 300 kcal/serving, from about 150 to about 250 kcal/serving, or about 200 kcal/serving.

In one embodiment, the nutritional composition, supplement, or fortifier is a nutritional supplement. The nutritional supplement may be a powder supplement for sprinkling on food or in a beverage.

In another embodiment, the nutritional composition, supplement or fortifier is a nutritional supplement in the form of a liquid, gel or spoonable food.

The nutritional supplement may comprise the L-arginine, L-citrulline, vitamins, minerals, and other components in any amounts suitable to enhance a subject's immune responses.

Suitably, the nutritional supplement comprises L-arginine in an amount of about 0.5 g/serving or more. Suitably, the nutritional supplement comprises L-citrulline in an amount of about 0.5 g/serving or more. Suitably, the nutritional supplement comprises: (a) L-arginine in an amount of about 0.5 g/serving or more; and (b) L-citrulline in an amount of about 0.5 g/serving or more.

Suitably, the nutritional supplement comprises: (a) vitamin A in an amount of about 200 μgRE/serving or more; (b) vitamin D2 in an amount of about 3 μg/serving or more; (c) vitamin E in an amount of about 2 mgTE/serving or more; (d) vitamin C in an amount of about 16 mg/serving or more; (e) vitamin B1 in an amount of about 0.3 mg/serving or more; (f) vitamin B2 in an amount of about 0.4 mg/serving or more; (g) niacin in an amount of about 3 mg/serving or more; (h) vitamin B6 in an amount of about 0.4 mg/serving or more; (i) folic acid in an amount of about 36 μg/serving or more; and (j) vitamin B12 in an amount of about 0.4 μg/serving or more.

Suitably, the nutritional supplement comprises: (a) potassium in an amount of about 250 mg/serving or more; (b) magnesium in an amount of about 33 mg/serving or more; (c) iron in an amount of about 5 mg/serving or more; (d) zinc in an amount of about 3 mg/serving or more; and (e) selenium in an amount of about 5 μg/serving or more.

The nutritional composition, supplement, or fortifier may be a nutritional composition, supplement, or fortifier for dietary management of a subject with an infection and/or having an immunodeficiency, in particular for dietary management of a subject with tuberculosis (TB).

In another aspect, the present invention provides a method of enhancing a subject's immune responses, the method comprising administering to the subject a nutritional composition, supplement, or fortifier according to the present invention.

In another aspect, the present invention provides a method of treating, preventing, and/or reducing the risk of occurrence of an infection in a subject, the method comprising administering to the subject a nutritional composition, supplement, or fortifier according to the present invention.

In another aspect, the present invention provides a method of preventing tuberculosis by keeping it in control in the latent state, the method comprising administering to the subject a nutritional composition, supplement, or fortifier according to the present invention.

In preferred embodiments, the infection is tuberculosis (TB). In more preferred embodiments, the infection is pulmonary TB.

In another aspect, the present invention provides a method of treating, preventing, and/or reducing the risk of occurrence of one or more symptoms of infection in a subject, the method comprising administering to the subject a nutritional composition, supplement, or fortifier according to the present invention. The symptoms of infection may be selected from: (i) positive sputum culture; (ii) cough; (iii) chest pain; (iv) fever; (v) night sweating; (vi) haemoptysis; (vii) dyspnea; and (viii) GI symptoms of diarrhea or constipation.

In another aspect, the present invention provides a method of accelerating wound healing in a subject, the method comprising administering to the subject a nutritional composition, supplement, or fortifier according to the present invention.

In another aspect, the present invention provides a method of treating or preventing chronic obstructive pulmonary disease (COPD) in a subject, the method comprising administering to the subject a nutritional composition, supplement, or fortifier according to the present invention.

In another aspect, the present invention provides L-citrulline for use in treating, preventing, and/or reducing the risk of occurrence of tuberculosis (TB) in a subject.

In another aspect, the present invention provides a method of treating or reducing the risk of tuberculosis (TB) in a subject, the method comprising administering to the subject a therapeutically effective amount of L-citrulline.

In another aspect, the present invention provides a method of reducing the occurrence of one or more symptoms of infection in a subject with tuberculosis (TB), comprising administering to the subject a therapeutically effective amount of L-citrulline, wherein the symptoms of infection are selected from: (i) positive sputum culture; (ii) cough; (iii) chest pain; (iv) fever (v) night sweating; (vi) haemoptysis (vii) dyspnea; and (viii) GI symptoms of diarrhea or constipation.

The L-citrulline may be administered to the subject by any suitable route and in any suitable dosage. Suitably, the L-citrulline is administered to the subject orally. Suitably, the L-citrulline is administered to the subject in an amount of about 0.5 g/day or more. Suitably, the L-citrulline is administered to the subject in the form of a nutritional composition, supplement, or fortifier, for example a nutritional composition, supplement, or fortifier as described above.

The L-citrulline may be administered in combination with L-arginine. The L-citrulline and L-arginine may be administered simultaneously. Suitably, the L-arginine is administered in an amount of about 0.5 g/day or more.

The L-citrulline may be administered to the subject as an adjunctive therapy, for example wherein the primary therapy is an antimicrobial therapy. One or more symptoms of infection may be reduced or prevented in the subject, for example wherein the symptoms of infection are selected from: (i) positive sputum culture; (ii) cough; (iii) chest pain; (iv) fever; (v) night sweating; (vi) haemoptysis; (vii) dyspnea, and (viii) GI symptoms of diarrhea or constipation.

In another aspect, the present invention provides a combination of L-arginine and L-citrulline for use in enhancing a subject's immune responses.

In another aspect, the present invention provides a combination of L-arginine and L-citrulline for use in treating, preventing, and/or reducing the risk of occurrence of an infection in a subject.

In another aspect, the present invention provides a combination of L-arginine and L-citrulline for use in accelerating wound healing in a subject.

In another aspect, the present invention provides a combination of L-arginine and L-citrulline for use in treating and/or preventing chronic obstructive pulmonary disease (COPD) in a subject.

In another aspect, the present invention provides a method of enhancing a subject's immune responses, the method comprising administering to the subject an effective amount of a combination of L-arginine and L-citrulline.

In another aspect, the present invention provides a method of treating or reducing the risk of infection in a subject, the method comprising administering to the subject an effective amount of a combination of L-arginine and L-citrulline.

In another aspect, the present invention provides a method of reducing the occurrence of one or more symptoms of infection in a subject, the method comprising administering to the subject an effective amount of a combination of L-arginine and L-citrulline, for example wherein the symptoms of infection are selected from: (i) positive sputum culture; (ii) cough; (iii) chest pain; (iv) fever; (v) night sweating; (vi) haemoptysis (vii) dyspnea; and (viii) GI symptoms of diarrhea or constipation.

In another aspect, the present invention provides a method accelerating wound healing in a subject, the method comprising administering to the subject an effective amount of a combination of L-arginine and L-citrulline.

In another aspect, the present invention provides a method of treating and/or preventing chronic obstructive pulmonary disease (COPD) in a subject, the method comprising administering to the subject an effective amount of a combination of L-arginine and L-citrulline. The combination may be administered to the subject by any suitable route and in any suitable dosage. The L-citrulline may be administered to the subject in an amount of about 0.5 g/day or more and/or the L-arginine may be administered to the subject in an amount of about 0.5 g/day or more. The combination of L-citrulline and L-arginine may be administered to the subject simultaneously. The combination may administered to the subject orally. The combination may be administered to the subject in the form of a nutritional composition, supplement, or fortifier, for example a nutritional composition, supplement, or fortifier as described above.

The subject may be any subject in need thereof. Suitably, the subject is an adult human. Suitably, the subject has or is at risk of an immunodeficiency.

### DESCRIPTION OF DRAWINGS

FIG. 1—Arginine synthesis and metabolic pathway

L-arginine is metabolized either by nitric oxide synthase (NOS) to NO and L-citrulline or it is hydrolyzed by arginase to L-ornithine and urea

FIGS. 2A and 2B—Arginine and Citrulline reduce intracellular Mtb load in macrophages

Both L-arginine and L-citrulline have been shown to reduce intracellular Mtb load in macrophages over 120 hours after infection compared to no treatment.

### DETAILED DESCRIPTION

Various preferred features and embodiments of the present invention will now be described by way of non-limiting examples.

It must be noted that as used herein and in the appended claims, the singular forms “a”, “an”, and “the” include plural referents unless the context clearly dictates otherwise.

The terms “comprising”, “comprises” and “comprised of” as used herein are synonymous with “including”, “includes”, “containing”, or “contains”, and are inclusive or open-ended and do not exclude additional, non-recited members, elements or steps. The terms “comprising”, “comprises” and “comprised of” also include the term “consisting of”.

Numeric ranges are inclusive of the numbers defining the range. As used herein the term “about” means approximately, in the region of, roughly, or around. When the term “about” is used in conjunction with a numerical value or range, it modifies that value or range by extending the boundaries above and below the numerical value(s) set forth. In general, the terms “about” and “approximately” are used herein to modify a numerical value(s) above and below the stated value(s) by 10%.

The publications discussed herein are provided solely for their disclosure prior to the filing date of the present application. Nothing herein is to be construed as an admission that such publications constitute prior art to the claims appended hereto.

This disclosure is not limited by the exemplary methods and materials disclosed herein, and any methods and materials similar or equivalent to those described herein can be used in the practice or testing of embodiments of this disclosure. The skilled person will understand that they can combine all features of the invention disclosed herein without departing from the scope of the invention as disclosed.

All publications mentioned in the specification are herein incorporated by reference.

Nutritional compositions, supplements and fortifiers

In one aspect, the present invention provides a nutritional composition, supplement, or fortifier comprising L-arginine, L-citrulline, a vitamin mixture, and a mineral mixture.

### L-Arginine

L-arginine (also known as 2-amino-5-guanidinopentanoic acid) is classified as a semi-essential or conditionally essential amino acid. Animal sources of L-arginine include meat, dairy products, and eggs and plant sources include seeds of all types, for example grains, beans, and nuts. L-arginine has the following formula:

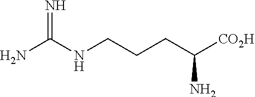 [SMILES: N=C(N)NCCC[C@H](N)C(=O)O]

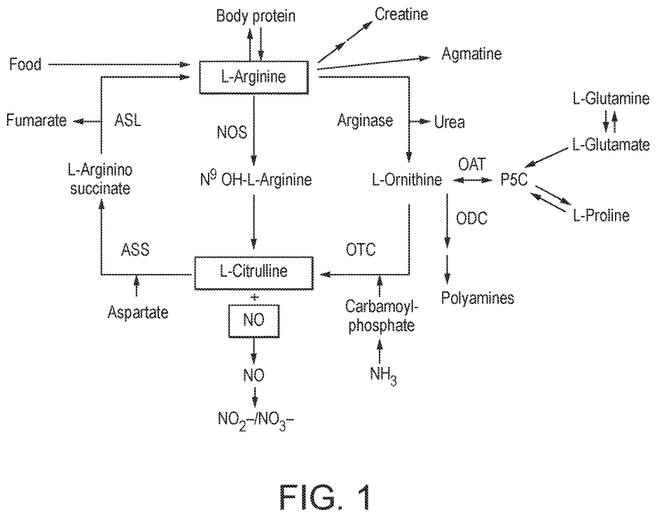

L-arginine is the precursor for the endogenous synthesis of nitric oxide (NO) due to the activity of NO synthase (NOS), which releases L-citrulline as a by-product (see FIG. 1). It has been shown that L-arginine is useful as an adjunctive therapy in patients with active tuberculosis, in which the effects are likely mediated by the increased production of NO (see Farazi, A., et al., 2015. Tuberculosis research and treatment, p. 205016).

The nutritional composition, supplement, or fortifier may be formulated such that the L-arginine is administered in a therapeutically effective dosage. One of skill in the art would be able to adjust the amount of L-arginine in the nutritional composition, supplement, or fortifier to achieve a desired dosage, for example as described in the section entitled “Routes of administration and dosage” below.

Suitably, L-arginine is present in an amount such that the nutritional composition, supplement, or fortifier provides L-arginine in a dosage of about 0.5 g/day or more, about 1.0 g/day or more, about 1.5 g/day or more, about 2.0 g/day or more, or about 2.5 g/day or more. Suitably, L-arginine is present in an amount such that the nutritional composition, supplement, or fortifier provides L-arginine in a dosage of about 10.0 g/day or less.

In some embodiments, L-arginine is present in an amount such that the nutritional composition, supplement, or fortifier provides L-arginine in a dosage of about 0.5 g/day or more. In some embodiments, L-arginine is present in an amount such that the nutritional composition, supplement, or fortifier provides L-arginine in a dosage of about 0.5 g/day.

In some embodiments, L-arginine is present in an amount such that the nutritional composition, supplement, or fortifier provides L-arginine in a dosage of about 1.0 g/day or more. In some embodiments, L-arginine is present in an amount such that the nutritional composition, supplement, or fortifier provides L-arginine in a dosage of about 1.0 g/day.

The nutritional composition, supplement, or fortifier may be formulated such that the L-arginine is administered in a suitable amount per serving. As used herein, a “serving” may refer to the recommended or typical amount of the product that is administered at one time. As used herein, “per serving” on food packaging may refer to the nutritional information provided on the label and how it relates to a single serving of the product.

Suitably, the nutritional composition, supplement, or fortifier comprises L-arginine in an amount of about 0.1 g/serving or more, about 0.2 g/serving or more, about 0.3 g/serving or more, about 0.4 g/serving or more, about 0.5 g/serving or more, about 1.0 g/serving or more, about 1.5 g/serving or more, about 2.0 g/serving or more, or about 2.5 g/serving or more. Suitably, the nutritional composition, supplement, or fortifier comprises L-arginine in an amount of about 10.0 g/serving or less.

In some embodiments, the nutritional composition, supplement, or fortifier comprises L-arginine in an amount of about 0.5 g/serving or more. In some embodiments, the nutritional composition, supplement, or fortifier comprises L-arginine in an amount of about 1.0 g/serving or more.

In some embodiments, the nutritional composition, supplement, or fortifier comprises L-arginine in an amount of about 0.5 g/serving. In some embodiments, the nutritional composition, supplement, or fortifier comprises L-arginine in an amount of about 1.0 g/serving.

Suitably, the nutritional composition, supplement, or fortifier comprises L-arginine in an amount of about 0.1 wt % or more, about 0.2 wt % or more, about 0.3 wt % or more, about 0.4 wt % or more, about 0.5 wt % or more, about 1.0 wt % or more, about 1.5 wt % or more, about 2.0 wt % or more, or about 2.5 wt % or more, for example on a dry weight basis. Suitably, the nutritional composition, supplement, or fortifier comprises L-arginine in an amount of about 10.0 wt % or less, for example on a dry weight basis.

Suitably, the nutritional composition, supplement, or fortifier comprises L-arginine in an amount of about 0.1 g/100 kcal or more, about 0.2 g/100 kcal or more, about 0.3 g/100 kcal or more, about 0.4 g/100 kcal or more, about 0.5 g/100 kcal or more, about 1.0 g/100 kcal or more, about 1.5 g/100 kcal or more, about 2.0 g/100 kcal or more, or about 2.5 g/100 kcal or more. Suitably, the nutritional composition, supplement, or fortifier comprises L-arginine in an amount of about 10.0 g/100 kcal or less.

### L-Citrulline

L-citrulline (also known as 2-amino-5-(carbamoylamino) pentanoic acid) is a non-protein building amino acid and is most abundant in melons. L-citrulline has the following formula:

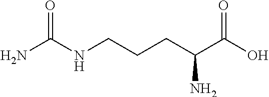 [SMILES: NC(=O)NCCC[C@H](N)C(=O)O]

It has been shown that L-citrulline supplementation increases plasma NO levels and reduces arginase activity in patients with type 2 diabetes (see Shatanawi, A., et al., 2020. Frontiers in pharmacology, 11, p. 584669). Moreover, supplemental L-citrulline may be more efficient than L-arginine in increasing systemic L-arginine availability (see Agarwal, U., et al., 2017. The Journal of nutrition, 147(4), pp. 596-602).

L-citrulline supplementation may also contribute to reversing the resistance of Mycobacteria to antibiotics. For example, exogenous citrulline has been shown to contribute to reverse the resistance of Salmonella to apramycin (see Yong, Y., et al., 2021. Frontiers in Microbiology, 12, p. 759170).

The nutritional composition, supplement, or fortifier may be formulated such that the L-citrulline is administered in a therapeutically effective dosage. One of skill in the art would be able to adjust the amount of L-citrulline in the nutritional composition, supplement, or fortifier to achieve a desired dosage, for example as described in the section entitled “Routes of administration and dosage” below.

Suitably, L-citrulline is present in an amount such that the nutritional composition, supplement, or fortifier provides L-citrulline in a dosage of about 0.5 g/day or more, about 1.0 g/day or more, about 1.5 g/day or more, about 2.0 g/day or more, or about 2.5 g/day or more. Suitably, L-citrulline is present in an amount such that the nutritional composition, supplement, or fortifier provides L-citrulline in a dosage of about 10.0 g/day or less.

In some embodiments, L-citrulline is present in an amount such that the nutritional composition, supplement, or fortifier provides L-citrulline in a dosage of about 0.5 g/day or more. In some embodiments, L-citrulline is present in an amount such that the nutritional composition, supplement, or fortifier provides L-citrulline in a dosage of about 0.5 g/day.

In some embodiments, L-citrulline is present in an amount such that the nutritional composition, supplement, or fortifier provides L-citrulline in a dosage of about 1.0 g/day or more. In some embodiments, L-citrulline is present in an amount such that the nutritional composition, supplement, or fortifier provides L-citrulline in a dosage of about 1.0 g/day.

The nutritional composition, supplement, or fortifier may be formulated such that the L-citrulline is administered in a suitable amount per serving.

Suitably, the nutritional composition, supplement, or fortifier comprises L-citrulline in an amount of about 0.1 g/serving or more, about 0.2 g/serving or more, about 0.3 g/serving or more, about 0.4 g/serving or more, about 0.5 g/serving or more, about 1.0 g/serving or more, about 1.5 g/serving or more, about 2.0 g/serving or more, or about 2.5 g/serving or more. Suitably, the nutritional composition, supplement, or fortifier comprises L-citrulline in an amount of about 10.0 g/serving or less.

In some embodiments, the nutritional composition, supplement, or fortifier comprises L-citrulline in an amount of about 0.5 g/serving or more. In some embodiments, the nutritional composition, supplement, or fortifier comprises L-citrulline in an amount of about 1.0 g/serving or more.

In some embodiments, the nutritional composition, supplement, or fortifier comprises L-citrulline in an amount of about 0.5 g/serving. In some embodiments, the nutritional composition, supplement, or fortifier comprises L-citrulline in an amount of about 1.0 g/serving.

Suitably, the nutritional composition, supplement, or fortifier comprises L-citrulline in an amount of about 0.1 wt % or more, about 0.2 wt % or more, about 0.3 wt % or more, about 0.4 wt % or more, about 0.5 wt % or more, about 1.0 wt % or more, about 1.5 wt % or more, about 2.0 wt % or more, or about 2.5 wt % or more, for example on a dry weight basis. Suitably, the nutritional composition, supplement, or fortifier comprises L-citrulline in an amount of about 10.0 wt % or less, for example on a dry weight basis.

Suitably, the nutritional composition, supplement, or fortifier comprises L-citrulline in an amount of about 0.1 g/100 kcal or more, about 0.2 g/100 kcal or more, about 0.3 g/100 kcal or more, about 0.4 g/100 kcal or more, about 0.5 g/100 kcal or more, about 1.0 g/100 kcal or more, about 1.5 g/100 kcal or more, about 2.0 g/100 kcal or more, or about 2.5 g/100 kcal or more. Suitably, the nutritional composition, supplement, or fortifier comprises L-citrulline in an amount of about 10.0 g/100 kcal or less.

### Combination of L-Arginine and L-Citrulline

The nutritional composition, supplement, or fortifier may be formulated such that the L-arginine and the L-citrulline are present in a therapeutically effective ratio. Suitably, the L-arginine and L-citrulline are present in a weight ratio of from about 0.1:1 to about 10:1, from about 0.2:1 to about 5:1, from about 0.25:1 to about 4:1, from about 0.3:1 to about 3:1, from about 0.5:1 to about 2:1, or about 1:1.

In some embodiments, the L-arginine and L-citrulline are present in a weight ratio of about 1:1.

Suitably, (a) L-arginine is present in an amount such that the nutritional composition, supplement, or fortifier provides L-arginine in a dosage of about 0.5 g/day or more and/or about 10.0 g/day or less; and (b) L-citrulline is present in an amount such that the nutritional composition, supplement, or fortifier provides L-citrulline in a dosage of about 0.5 g/day or more and/or about 10.0 g/day or less.

In some embodiments, (a) L-arginine is present in an amount such that the nutritional composition, supplement, or fortifier provides L-arginine in a dosage of about 0.5 g/day or more; and (b) L-citrulline is present in an amount such that the nutritional composition, supplement, or fortifier provides L-citrulline in a dosage of about 0.5 g/day or more.

In some embodiments, (a) L-arginine is present in an amount such that the nutritional composition, supplement, or fortifier provides L-arginine in a dosage of about 1.0 g/day or more; and (b) L-citrulline is present in an amount such that the nutritional composition, supplement, or fortifier provides L-citrulline in a dosage of about 1.0 g/day or more.

In some embodiments, (a) L-arginine is present in an amount such that the nutritional composition, supplement, or fortifier provides L-arginine in a dosage of about 0.5 g/day; and (b) L-citrulline is present in an amount such that the nutritional composition, supplement, or fortifier provides L-citrulline in a dosage of about 0.5 g/day.

In some embodiments, (a) L-arginine is present in an amount such that the nutritional composition, supplement, or fortifier provides L-arginine in a dosage of about 1.0 g/day; and (b) L-citrulline is present in an amount such that the nutritional composition, supplement, or fortifier provides L-citrulline in a dosage of about 1.0 g/day.

Suitably, the nutritional composition, supplement, or fortifier comprises: (a) L-arginine in an amount of about 0.1 g/serving or more and/or about 10.0 g/serving or less; and (b) L-citrulline in an amount of about 0.1 g/serving or more and/or about 10.0 g/serving or less.

In some embodiments, the nutritional composition, supplement, or fortifier comprises: (a) L-arginine in an amount of about 0.5 g/serving or more; and (b) L-citrulline in an amount of about 0.5 g/serving or more.

In some embodiments, the nutritional composition, supplement, or fortifier comprises: (a) L-arginine in an amount of about 1.0 g/serving or more; and (b) L-citrulline in an amount of about 1.0 g/serving or more.

In some embodiments, the nutritional composition, supplement, or fortifier comprises: (a) L-arginine in an amount of about 0.5 g/serving; and (b) L-citrulline in an amount of about 0.5 g/serving.

In some embodiments, the nutritional composition, supplement, or fortifier comprises: (a) L-arginine in an amount of about 1.0 g/serving; and (b) L-citrulline in an amount of about 1.0 g/serving.

Suitably, the nutritional composition, supplement, or fortifier comprises: (a) L-arginine in an amount of about 0.1 wt % or more and/or about 10.0 wt % or less, for example on a dry weight basis; and (b) L-citrulline in an amount of about 0.1 wt % or more and/or about 10.0 wt % or less, for example on a dry weight basis.

Suitably, the nutritional composition, supplement, or fortifier comprises: (a) L-arginine in an amount of about 0.1 g/100 kcal or more and/or about 10.0 g/100 kcal or less; and (b) L-citrulline in an amount of about 0.1 g/100 kcal or more and/or about 10.0 g/100 kcal or less.

### Vitamin Mixture

As used herein, a “vitamin mixture” may refer to a mixture of one or more vitamins. Vitamins are organic micronutrients required by the body to carry out a range of normal functions and include vitamin A, vitamin D2, vitamin E, vitamin K1, vitamin C, vitamin B1, vitamin B2, niacin (or vitamin B3), pantothenic acid (or vitamin B5), vitamin B6, folic acid (or vitamin B9), vitamin B12, and biotin (or vitamin B7).

Preferably, the vitamin mixture comprises one or more vitamins that enhance a subject's immune responses. Some major vitamins like vitamins A, B, C, D, and E have been reported to boost the immune system by strengthening the activity of immune cells during any pathogen attack (see e.g. Aslam, F., et al., 2017. Vitamins & Minerals, 6(1), p. 1000153).

Suitably, the vitamin mixture may comprise one or more vitamin selected from: vitamin A, vitamin D2, vitamin E, vitamin C, vitamin B1, vitamin B2, niacin, vitamin B6, folic acid, and vitamin B12.

In some embodiments, the vitamin mixture comprises two or more, three or more, four or more, five or more, six or more, seven or more, eight or more, or nine or more vitamins selected from: vitamin A, vitamin D2, vitamin E, vitamin C, vitamin B1, vitamin B2, niacin, vitamin B6, folic acid, and vitamin B12.

In some embodiments, the vitamin mixture comprises or consists of vitamin A, vitamin D2, vitamin E, vitamin C, vitamin B1, vitamin B2, niacin, vitamin B6, folic acid, and vitamin B12.

In some embodiments, the vitamin mixture comprises or consists of vitamin A, vitamin D2, vitamin E, vitamin K1, vitamin C, vitamin B1, vitamin B2, niacin, pantothenic acid, vitamin B6, folic acid, vitamin B12, and biotin.

The nutritional composition, supplement, or fortifier may be formulated such that the vitamins are administered in a therapeutically effective dosage. One of skill in the art would be able to adjust the amount of vitamins in the nutritional composition, supplement, or fortifier to achieve a desired dosage. Suitable dosages are known in the art and may be expressed as a “Recommended Dietary Allowance” or “RDA”.

The RDA may refer to the average daily level of intake sufficient to meet the nutrient requirements of nearly all healthy individuals (e.g. 97-98% of individuals) and is typically used to plan nutritionally adequate diets for individuals (see e.g. Recommended Dietary Allowances: 10th Edition. Washington (DC): National Academies Press (US); 1989).

Suitably, each vitamin is present in an amount such that the nutritional composition, supplement, or fortifier provides the vitamin in a dosage of about 10% RDA/day or more, about 20% RDA/day or more, about 30% RDA/day or more, about 40% RDA/day or more, about 50% RDA/day or more, about 60% RDA/day or more, about 70% RDA/day or more, about 80% RDA/day or more. Suitably, each vitamin is present in an amount such that the nutritional composition, supplement, or fortifier provides each vitamin in a dosage of about 100% RDA/day or less.

In some embodiments, each vitamin is present in an amount such that the nutritional composition, supplement, or fortifier provides each vitamin in a dosage of about 50% RDA/day or more.

Suitably, the nutritional composition, supplement, or fortifier comprises each vitamin in an amount of about 10% RDA/serving or more, about 20% RDA/serving or more, about 30% RDA/serving or more, about 40% RDA/serving or more, about 50% RDA/serving or more, about 60% RDA/serving or more, about 70% RDA/serving or more, or about 80% RDA/serving or more. Suitably, the nutritional composition, supplement, or fortifier comprises each vitamin in an amount of about 100% RDA/serving or less.

In some embodiments, the nutritional composition, supplement, or fortifier comprises each vitamin in an amount of about 40% RDA/serving or more.

### Vitamin A

In some embodiments, the vitamin mixture comprises vitamin A.

Vitamin A may refer to the fat-soluble compound all-trans-retinol and is also commonly used to refer to retinol and its active metabolites, including retinal, retinyl ester, and retinoic acid. Vitamin A is known as an anti-inflammation vitamin because of its critical role in enhancing immune function, is involved in the development of the immune system and plays regulatory roles in cellular immune responses and humoral immune processes. Vitamin A has demonstrated a therapeutic effect in the treatment of various infectious diseases (see e.g. Huang, Z., et al., 2018. Role of vitamin A in the immune system. Journal of clinical medicine, 7(9), p. 258).

Suitable dosages of vitamin A will be well known to those of skill in the art. For example, the RDA of vitamin A may be from about 500 μgRE to about 1500 μgRE. For example, the RDA of vitamin A may be about 1000 μgRE. “RE” may refer to retinol equivalents, where 1 μg retinol=6 μg β-carotene=12 μg other active carotenoids.

### Vitamin D

In some embodiments, the vitamin mixture comprises vitamin D. In some embodiments, the vitamin mixture comprises vitamin D2.

Several forms of vitamin D exist. The two major forms are vitamin D2 and vitamin D3. Vitamin D2 is commonly known as ergocalciferol and vitamin D3 as cholecalciferol. Vitamin D plays an important part in regulating human immune response and is known for its activity in the expression of genes that produce proteins which are toxic to a number of foreign microbes (see e.g. Mora, J. R., et al., 2008. Nature reviews immunology, 8(9), pp. 685-698).

Suitable dosages of vitamin D (e.g. vitamin D2) will be well known to those of skill in the art. For example, the RDA of vitamin D2 may be from about 10 μg to about 20 μg. For example, the RDA of vitamin D2 may be about 15 μg.

### Vitamin E

In some embodiments, the vitamin mixture comprises vitamin E.

Vitamin E may refer to a group of eight fat soluble compounds that include four tocopherols and four tocotrienols (alpha, beta, gamma, delta). Vitamin E deficiency has been demonstrated to impair normal functions of the immune system in animals and humans, which can be corrected by vitamin E repletion (see e.g. Lewis, E. D., et al., 2019. IUBMB life, 71(4), pp. 487-494).

Suitable dosages of vitamin E will be well known to those of skill in the art. For example, the RDA of vitamin E may be from about 5 μgTE to about 15 μgTE. For example, the RDA of vitamin E may be about 10 μgTE. The nutritional content of vitamin E may be defined by equivalency to α-tocopherol activity.

### Vitamin C

In some embodiments, the vitamin mixture comprises vitamin C.

Vitamin C (also known as ascorbic acid and ascorbate) is a water-soluble vitamin found in citrus and other fruits and vegetables. Vitamin C contributes to immune defence by supporting various cellular functions of both the innate and adaptive immune system (see e.g. Carr, A. C. and Maggini, S., 2017. Nutrients, 9(11), p. 1211).

Suitable dosages of vitamin C will be well known to those of skill in the art. For example, the RDA of vitamin C may be from about 60 mg to about 100 mg For example, the RDA of vitamin C may be about 80 mg.

### Vitamin B1

In some embodiments, the vitamin mixture comprises vitamin B1.

Vitamin B1 (also known as thiamine) is required by the body for the efficient breakdown of carbohydrates. It is present in an array of food products including grains, nuts, beans, yeast, and meat. It is known that vitamin B1 has a protective effect against oxidative retinal damage induced by anti-tuberculosis drugs and may help to limit Mycobacterium tuberculosis growth via regulating innate immunity (Hu, S., et al., 2018. Frontiers in immunology, 9, p. 1778).

Suitable dosages of vitamin B1 will be well known to those of skill in the art. For example, the RDA of vitamin B1 may be from about 1.0 mg to about 2.0 mg. For example, the RDA of vitamin B1 may be about 1.4 mg.

### Vitamin B2

In some embodiments, the vitamin mixture comprises vitamin B2.

Vitamin B2 (also known as riboflavin) is essential to the formation of two major coenzymes, flavin mononucleotide and flavin adenine dinucleotide. These coenzymes are involved in energy metabolism, cellular respiration, and antibody production, as well as normal growth and development. Vitamin B2 plays a role in oxidative metabolism and has anti-inflammatory properties (see e.g. Mikkelsen, K. and Apostolopoulos, V., 2019. Nutrition and immunity, pp. 115-125).

Suitable dosages of vitamin B2 will be well known to those of skill in the art. For example, the RDA of vitamin B2 may be from about 1.0 mg to about 2.5 mg For example, the RDA of vitamin B2 may be about 2.0 mg.

### Vitamin B3

In some embodiments, the vitamin mixture comprises vitamin B3. In some embodiments, the vitamin mixture comprises niacin

Vitamin B3 may refer to a vitamin family that includes three forms or vitamers: niacin (nicotinic acid), nicotinamide (niacinamide), and nicotinamide riboside. All three forms are converted within the body to nicotinamide adenine dinucleotide (NAD), which is a coenzyme central to metabolism. Niacin has important modulatory effects on the production of inflammatory mediators, and the migration of immune cells (see e.g. Spinas, E., et al., 2015. J Biol Regul Homeost Agents, 29(2), pp. 283-8).

Suitable dosages of vitamin B3 (e.g. niacin) will be well known to those of skill in the art. For example, the RDA of vitamin B3 (e.g. niacin) may be from about 10 mg to about 20 mg. For example, the RDA of vitamin B3 (e.g. niacin) may be about 14 mg.

### Vitamin B6

In some embodiments, the vitamin mixture comprises vitamin B6.

Vitamin B6 may refer to pyridoxine, pyridoxal, pyridoxamine, and their respective phosphorylated derivatives. Pyridoxal 5′-phosphate is the metabolically active form of vitamin B6 and serves as a co-factor in many cellular reactions. Experimental human and animal model indicate that vitamin B6 deficiency influences both innate and adaptive immunity, and decreased number, function, and proliferation of immune cells, including T lymphocytes, have been reported (see e.g. Spinas, E., et al., 2015. J Biol Regul Homeost Agents, 29(2), pp. 283-8).

Suitable dosages of vitamin B6 will be well known to those of skill in the art. For example, the RDA of vitamin B6 may be from about 1.0 mg to about 2.5 mg. For example, the RDA of vitamin B6 may be about 1.9 mg.

### Folic Acid

In some embodiments, the vitamin mixture comprises folic acid.

Folic acid (also known as vitamin B9 and folacin) is required for the body to make DNA and RNA and metabolise amino acids necessary for cell division. Supplementation of folic acid has been shown to improve immune response in both animal and human models (see e.g. Mikkelsen, K. and Apostolopoulos, V., 2019. Nutrition and immunity, pp. 103-114).

Suitable dosages of folic acid will be well known to those of skill in the art. For example, the RDA of folic acid may be from about 100 μg to about 400 μg. For example, the RDA of folic acid may be about 180 μg

### Vitamin B12

In some embodiments, the vitamin mixture comprises vitamin B12.

Vitamin B12 (also known as cobalamin) is a water-soluble vitamin involved in metabolism. Supplementation of vitamin B12 has been shown to improve immune response in both animal and human models (see e.g. Mikkelsen, K. and Apostolopoulos, V., 2019. Nutrition and immunity, pp. 103-114).

Suitable dosages of vitamin B12 will be well known to those of skill in the art. For example, the RDA of vitamin B12 may be from about 1.5 mg to about 3 mg. For example, the RDA of vitamin B12 may be about 2.2 mg.

### Mineral Mixture

As used herein, a “mineral mixture” may refer to a mixture of one or more minerals. Minerals are elements required by the body to carry out a range of normal functions and include calcium, phosphorous, iodine, iron, magnesium, copper, zinc, manganese, chlorine, potassium, sodium, selenium, chromium, molybdenum. Minerals are usually added in salt form.

Preferably, the mineral mixture comprises one or more minerals that enhance a subject's immune responses. Some minerals like iron, zinc, copper, selenium, and magnesium have been reported to play important roles in immune responses (see e.g. Gombart, A. F., et al. Nutrients, 12(1), p. 236).

Suitably, the mineral mixture may comprise one or more mineral selected from: potassium, magnesium, iron, zinc, and selenium.

In some embodiments, the mineral mixture comprises two or more, three or more, or four or more minerals selected from: potassium, magnesium, iron, zinc, and selenium.

In some embodiments, the mineral mixture comprises or consists of potassium, magnesium, iron, zinc, and selenium.

In some embodiments, the mineral mixture comprises or consists of potassium, magnesium, iron, zinc, selenium, sodium, calcium, phosphorus, copper, manganese, iodine, chromium, and molybdenum.

The nutritional composition, supplement, or fortifier may be formulated such that the minerals are administered in a therapeutically effective dosage. One of skill in the art would be able to adjust the amount of minerals in the nutritional composition, supplement, or fortifier to achieve a desired dosage.

Suitably, each mineral is present in an amount such that the nutritional composition, supplement, or fortifier provides the mineral in a dosage of about 10% RDA/day or more, about 15% RDA/day or more, about 20% RDA/day or more, about 25% RDA/day or more, about 30% RDA/day or more, about 40% RDA/day or more, about 50% RDA/day or more, about 60% RDA/day or more, about 70% RDA/day or more, or about 80% RDA/day or more.

Suitably, each mineral is present in an amount such that the nutritional composition, supplement, or fortifier provides each mineral in a dosage of about 100% RDA/day or less.

In some embodiments, each mineral is present in an amount such that the nutritional composition, supplement, or fortifier provides each mineral in a dosage of about 15% RDA/day or more.

In some embodiments, potassium and magnesium are present in an amount such that the nutritional composition, supplement, or fortifier provides potassium and magnesium each in a dosage of about 15% RDA/day or more.

In some embodiments, iron, zinc, and selenium are present in an amount such that the nutritional composition, supplement, or fortifier provides iron, zinc, and selenium each in a dosage of about 30% RDA/day or more.

Suitably, the nutritional composition, supplement, or fortifier comprises each mineral in an amount of about 10% RDA/serving or more, about 15% RDA/serving or more, about 20% RDA/serving or more, about 25% RDA/serving or more, about 30% RDA/serving or more, about 35% RDA/serving or more, or about 40% RDA/serving or more. Suitably, the nutritional composition, supplement, or fortifier comprises each mineral in an amount of about 100% RDA/serving or less.

In some embodiments, the nutritional composition, supplement, or fortifier comprises each mineral in an amount of about 15% RDA/serving or more.

In some embodiments, the nutritional composition, supplement, or fortifier comprises potassium and magnesium in an amount of about 15% RDA/serving or more.

In some embodiments, the nutritional composition, supplement, or fortifier comprises iron, zinc and selenium in an amount of about 30% RDA/serving or more.

### Potassium

In some embodiments, the mineral mixture comprises potassium.

Suitable dosages of potassium will be well known to those of skill in the art. For example, the RDA of potassium may be from about 2500 mg to about 4000 mg. For example, the RDA of potassium may be about 3500 mg.

### Magnesium

In some embodiments, the mineral mixture comprises magnesium.

Magnesium plays a role in innate and acquired immune responses, and is involved in synthesis of immunoglobulin as cofactor, immune cell adherence, antibody dependent cytosis, and T-helper B-cell adherence. Epidemiological data have shown that higher intake of magnesium is associated with lower incidence of respiratory problems (see e.g. Agrawal, Y., et al., 2017. Journal of clinical and diagnostic research: JCDR, 11(6), p.BC01).

Suitable dosages of magnesium will be well known to those of skill in the art. For example, the RDA of magnesium may be from about 350 mg to about 500 mg. For example, the RDA of magnesium may be about 440 mg.

### Iron

In some embodiments, the mineral mixture comprises iron.

Both iron deficiency and overload exist in TB patients and may contribute to disease progression and poor clinical outcomes. Strategies to maintain normal iron status in TB patients could be helpful to reduce TB morbidity and mortality (see e.g. Isanaka, S., et al., 2012. PloS one, 7(5), p.e37350)

Suitable dosages of iron will be well known to those of skill in the art. For example, the RDA of iron may be from about 10 mg to about 40 mg. For example, the RDA of iron may be about 29 mg.

### Zinc

In some embodiments, the mineral mixture comprises zinc.

Zinc is a micronutrient that is critical for the proper functioning of the immune system and plays an important role in the fight against TB. Zinc supplementation has been shown to result in earlier sputum smear conversion in the micronutrient group during the first 6 weeks (see e.g. Zolfaghari, B., et al., 2021. Reports of biochemistry & molecular biology, 10(2), p. 173) Suitable dosages of zinc will be well known to those of skill in the art. For example, the RDA of zinc may be from about 10 mg to about 20 mg. For example, the RDA of zinc may be about 17 mg.

### Selenium

In some embodiments, the mineral mixture comprises selenium.

Selenium status may affect the function of cells of both adaptive and innate immunity. Four studies have reported significantly decreased serum selenium concentrations in patients with TB compared with healthy controls (see e.g. Steinbrenner, H., et al., 2015. Advances in nutrition, 6(1), pp. 73-82).

Suitable dosages of selenium will be well known to those of skill in the art. For example, the RDA of selenium may be from about 30 μg to about 60 μg. For example, the RDA of selenium may be about 40 μg.

### Antioxidants

In some embodiments, the nutritional composition, supplement, or fortifier of the invention may comprise any antioxidants.

An antioxidant is a molecule that can slow or stop the oxidation, or electron transfer, of other molecules. Antioxidants can be found in food. Antioxidants are molecules that relieve oxidative stress by preventing the formation and oxidation of free radicals.

Suitable antioxidants include one or more of: glutathione, coenzyme Q10, lipoic acid, flavonoids, phenols, polyphenols, and phytoestrogens. In some embodiments, the antioxidant is present in an amount that the nutritional composition, supplement, or fortifier provides a dosage of about 15% RDA/day or more. In some embodiments, the antioxidant is present in an amount that the nutritional composition, supplement, or fortifier provides a dosage of about 30% RDA/day or more. The recommended daily dose is equivalent to 8-11,000 antioxidant units a day.

### Fibers

In some embodiments, the nutritional composition, supplement, or fortifier of the invention may comprise any fibers.

Dietary fiber is the edible parts of plants or analogous carbohydrates that are resistant to digestion and absorption in the human small intestine, with complete or partial fermentation in the large intestine. Dietary fiber includes polysaccharides, oligosaccharides, lignin, and associated plant substances. Dietary fibers promote beneficial physiologic effects including in the gastrointestinal tract including relief from diarrhea and constipation as well as blood cholesterol and/or blood glucose attenuation.

The nutritional composition, supplement, or fortifier may further comprise any mixture that is suitable to enhance a subject's gastrointestinal health including fibers. Suitable fibers include one or more of: soluble fiber, insoluble fiber, inulin, or vegetable gums.

Soluble or insoluble fiber may be beneficial for alleviating symptoms of diarrhea or constipation and gastrointestinal discomfort.

Inulin and oligosaccharides may contribute to anti-inflammatory actions upon the bowel. They may increase absorption of calcium, magnesium, and iron.

Vegetable gum fiber such as guar gum and gum Arabic may further contribute to alleviating symptoms of gastrointestinal discomfort.

In some embodiments, the fiber is present in an amount that the nutritional composition, supplement, or fortifier provides a dosage of about 15% RDA/day or more. In some embodiments, the fiber is present in an amount that the nutritional composition, supplement, or fortifier provides a dosage of about 30% RDA/day or more. The recommended daily dosage is equivalent to 20 g to 30 g food fiber per day.

### Other Components

The nutritional composition, supplement, or fortifier of the invention may comprise any other suitable components. In particular, components which contribute to a subject's normal nutrition or which enhance a subject's immune responses.

Suitably, the nutritional composition, supplement, or fortifier further comprises one or more macronutrient, such as protein, carbohydrate, or lipids. In some embodiments, the nutritional composition, supplement, or fortifier comprises protein. In some embodiments, the nutritional composition, supplement, or fortifier comprises carbohydrate. In some embodiments, the nutritional composition, supplement, or fortifier comprises lipid. In some embodiments, the nutritional composition, supplement, or fortifier comprises protein and carbohydrate. In some embodiments, the nutritional composition, supplement, or fortifier comprises protein, carbohydrate, and lipid.

The nutritional composition, supplement, or fortifier of the invention may also contain other substances which may have a beneficial effect such as lactoferrin, osteopontin, TGFbeta, sIgA, glutamine, nucleotides, nucleosides, and the like.

### Nutritional Compositions

In one aspect, the present invention provides a nutritional composition comprising L-arginine, L-citrulline, a vitamin mixture, and a mineral mixture.

A “nutritional composition” may refer to a composition which nourishes a subject. The nutritional composition may be prepared in any suitable manner. The nutritional composition is not particularly limited as long as it is suitable for administration (e.g. oral, enteral, or parenteral administration). Examples of suitable nutritional compositions include powder formulas, foodstuffs, drinks, tube feeds, and drug bases.

The nutritional composition according to the invention may be a food for dietary management of a subject with disease related malnutrition. In particular, wherein the disease is an infection such as TB.

The nutritional composition according to the invention may be a food for dietary management of a subject with an infection and/or having an immunodeficiency. In particular, the nutritional composition according to the invention may be a food for dietary management of a subject with TB.

The nutritional composition according to the invention may be provided in the form of a powder. Powder formulas can be reconstituted with liquids such as water or milk prior to ingestion. In some embodiments, the powder is for reconstitution with water. In some embodiments, the powder is for reconstitution with milk. By reconstituting in milk it may be possible to add less vitamins, make the product better tasting, and save costs.

The nutritional composition according to the invention may be provided in the form of a liquid, gel or spoonable food.

The nutritional composition according to the invention may be nutritionally complete. “Nutritionally complete” food may provide all the all the carbohydrates, fats, protein, vitamins and minerals necessary to nourish a subject.

Suitably, in the context of a nutritional composition (e.g. in the form of a powder), a “serving” may be from about 20 g to about 100 g. In some embodiments, a “serving” in the context of a nutritional composition (e.g. in the form of a powder) is about 50 g.

### L-Arginine

The nutritional composition may comprise L-arginine in any therapeutically effective amount.

Suitably, the nutritional composition comprises L-arginine in an amount of about 0.5 g/serving or more and/or about 10.0 g/serving or less. In some embodiments, the nutritional composition comprises L-arginine in an amount of about 1.0 g/serving or more. In some embodiments, the nutritional composition comprises L-arginine in an amount of about 1.0 g/serving.

Suitably, the nutritional composition comprises L-arginine in an amount of about 1.0 wt % or more and/or about 20.0 wt % or less. In some embodiments, the nutritional composition comprises L-arginine in an amount of about 2.0 wt % or more. In some embodiments, the nutritional composition comprises L-arginine in an amount of about 2.0 wt %. This may be on a dry weight basis.

Suitably, the nutritional composition comprises L-arginine in an amount of about 0.25 g/100 kcal or more and/or about 5.0 g/100 kcal or less. In some embodiments, the nutritional composition comprises L-arginine in an amount of about 0.5 g/100 kcal or more. In some embodiments, the nutritional composition comprises L-arginine in an amount of about 0.5 g/100 kcal.

### L-Citrulline

The nutritional composition may comprise L-citrulline in any therapeutically effective amount.

Suitably, the nutritional composition comprises L-citrulline in an amount of about 0.5 g/serving or more and/or about 10.0 g/serving or less. In some embodiments, the nutritional composition comprises L-citrulline in an amount of about 1.0 g/serving or more. In some embodiments, the nutritional composition comprises L-citrulline in an amount of about 1.0 g/serving. In some embodiments, the nutritional composition comprises L-citrulline in an amount of about 1.25 g/serving or more. In some embodiments, the nutritional composition comprises L-citrulline in an amount of about 1.25 g/serving.

Suitably, the nutritional composition comprises L-citrulline in an amount of about 1.0 wt % or more and/or about 20.0 wt % or less. In some embodiments, the nutritional composition comprises L-citrulline in an amount of about 2.5 wt % or more. In some embodiments, the nutritional composition comprises L-citrulline in an amount of about 2.5 wt %. This may be on a dry weight basis.

Suitably, the nutritional composition comprises L-citrulline in an amount of about 0.25 g/100 kcal or more and/or about 5.0 g/100 kcal or less. In some embodiments, the nutritional composition comprises L-citrulline in an amount of about 0.6 g/100 kcal or more. In some embodiments, the nutritional composition comprises L-citrulline in an amount of about 0.6 g/100 kcal.

### Combination of L-Arginine and L-Citrulline

In some embodiments, the nutritional composition comprises: (a) L-arginine in an amount of about 1.0 g/serving or more; and (b) L-citrulline in an amount of about 1.0 g/serving or more.

In some embodiments, the nutritional composition comprises: (a) L-arginine in an amount of about 1.0 g/serving or more; and (b) L-citrulline in an amount of about 1.25 g/serving or more.

In some embodiments, the nutritional composition comprises: (a) L-arginine in an amount of about 1.0 g/serving; and (b) L-citrulline in an amount of about 1.0 g/serving.

In some embodiments, the nutritional composition comprises: (a) L-arginine in an amount of about 1.0 g/serving; and (b) L-citrulline in an amount of about 1.25 g/serving.

In some embodiments, the nutritional composition comprises: (a) L-arginine in an amount of about 2.0 wt % or more; and (b) L-citrulline in an amount of about 2.5 wt % or more, for example on a dry weight basis.

In some embodiments, the nutritional composition comprises: (a) L-arginine in an amount of about 2.0 wt %; and (b) L-citrulline in an amount of about 2.5 wt %, for example on a dry weight basis.

In some embodiments, the nutritional composition comprises: (a) L-arginine in an amount of about 0.5 g/100 kcal or more; and (b) L-citrulline in an amount of about 0.6 g/100 kcal or more.

In some embodiments, the nutritional composition comprises: (a) L-arginine in an amount of about 0.5 g/100 kcal; and (b) L-citrulline in an amount of about 0.6 g/100 kcal.

### Vitamin Mixture

The nutritional composition may comprise the vitamins in any therapeutically effective amount. Suitably, the nutritional composition comprises:
(a) vitamin A in an amount of about 300 μgRE/serving or more, about 400 μgRE/serving or more, about 500 μgRE/serving or more, or about 600 μgRE/serving or more;
(b) vitamin D2 in an amount of about 5 μg/serving or more, about 6 μg/serving or more, about 7 μg/serving or more, about 8 μg/serving or more, about 9 μg/serving or more, or about 10 μg/serving or more;
(c) vitamin E in an amount of about 3.0 mgTE/serving or more, about 3.5 mgTE/serving or more, about 4.0 mgTE/serving or more, about 4.5 mgTE/serving or more, about 5.0 mgTE/serving or more, about 5.5 mgTE/serving or more, about 6.0 mgTE/serving or more, or about 6.5 mgTE/serving or more;
(d) vitamin C in an amount of about 25 mg/serving or more, about 30 mg/serving or more, about 35 mg/serving or more, about 40 mg/serving or more, about 45 mg/serving or more, or about 50 mg/serving or more;
(e) vitamin B1 in an amount of about 0.5 mg/serving or more, about 0.6 mg/serving or more, about 0.7 mg/serving or more, about 0.8 mg/serving or more, about 0.9 mg/serving or more, or about 1.0 mg/serving or more;
(f) vitamin B2 in an amount of about 0.7 mg/serving or more, about 0.8 mg/serving or more, about 0.9 mg/serving or more, about 1.0 mg/serving or more, about 1.1 mg/serving or more, about 1.2 mg/serving or more, about 1.3 mg/serving or more, or about 1.4 mg/serving or more;
(g) niacin in an amount of about 5 mg/serving or more, about 6 mg/serving or more, about 7 mg/serving or more, about 8 mg/serving or more, about 9 mg/serving or more, or about 10 mg/serving or more;
(h) vitamin B6 in an amount of about 0.6 mg/serving or more, about 0.7 mg/serving or more, about 0.8 mg/serving or more, about 0.9 mg/serving or more, about 1.0 mg/serving or more, about 1.1 mg/serving or more, or about 1.2 mg/serving or more;
(i) folic acid in an amount of about 60 μg/serving or more, about 70 μg/serving or more, about 80 μg/serving or more, about 90 μg/serving or more, about 100 μg/serving or more, about 110 μg/serving or more, or about 120 μg/serving or more; and/or
(j) vitamin B12 in an amount of about 0.7 μg/serving or more, about 0.8 μg/serving or more, about 0.9 μg/serving or more, about 1.0 μg/serving or more, about 1.1 μg/serving or more, about 1.2 μg/serving or more, about 1.3 μg/serving or more, or about 1.4 μg/serving or more.

In some embodiments, the nutritional composition further comprises:
(k) vitamin K1 in an amount of about 6 μg/serving or more, about 7 μg/serving or more, about 8 μg/serving or more, about 9 μg/serving or more, about 10 μg/serving or more, about 11 μg/serving or more, or about 12 μg/serving or more;
(l) pantothenic acid an amount of about 0.35 mg/serving or more, about 0.40 mg/serving or more, about 0.45 mg/serving or more, about 0.50 mg/serving or more, about 0.55 mg/serving or more, about 0.60 mg/serving or more, about 0.65 mg/serving or more, about 0.70 mg/serving or more, or about 0.75 mg/serving or more; and/or
(m) biotin an amount of about 2.0 μg/serving or more, about 2.5 μg/serving or more, about 3.0 μg/serving or more, about 3.5 μg/serving or more, about 4.0 μg/serving or more, or about 4.5 μg/serving or more.

In some embodiments, the nutritional composition comprises: (a) vitamin A in an amount of about 250 μgRE/100 kcal or more; (b) vitamin D2 in an amount of about 4 μg/100 kcal or more; (c) vitamin E in an amount of about 3.0 mgTE/100 kcal or more; (d) vitamin C in an amount of about 20 mg/100 kcal or more; (e) vitamin B1 in an amount of about 0.4 mg/100 kcal or more; (f) vitamin B2 in an amount of about 0.6 mg/100 kcal or more; (g) niacin in an amount of about 4 mg/100 kcal or more; (h) vitamin B6 in an amount of about 0.5 mg/100 kcal or more; (i) folic acid in an amount of about 50 μg/100 kcal or more; and/or (j) vitamin B12 in an amount of about 0.6 μg/100 kcal or more.

In some embodiments, the nutritional composition further comprises: (k) vitamin K1 in an amount of about 5 μg/100 kcal or more; (l) pantothenic acid an amount of about 0.3 mg/100 kcal or more; and/or (m) biotin an amount of about 2 μg/100 kcal or more.

### Mineral Mixture

The nutritional composition may comprise the minerals in any therapeutically effective amount. Suitably, the nutritional composition comprises:
(a) potassium in an amount of about 250 mg/serving or more, about 300 mg/serving or more, about 350 mg/serving or more, about 400 mg/serving or more, about 450 mg/serving or more, or about 500 mg/serving or more;
(b) magnesium in an amount of about 30 mg/serving or more, about 35 mg/serving or more, about 40 mg/serving or more, about 45 mg/serving or more, about 50 mg/serving or more, about 55 mg/serving or more, about 60 mg/serving or more, or about 65 mg/serving or more;
(c) iron in an amount of about 4 mg/serving or more, about 5 mg/serving or more, about 6 mg/serving or more, about 7 mg/serving or more, or about 8 mg/serving or more;
(d) zinc in an amount of about 2 mg/serving or more, about 3 mg/serving or more, about 4 mg/serving or more, or about 5 mg/serving or more; and/or
(e) selenium in an amount of about 6 μg/serving or more, about 7 μg/serving or more, about 8 μg/serving or more, about 9 μg/serving or more, about 10 μg/serving or more, about 11 μg/serving or more, or about 12 μg/serving or more.

In some embodiments, the nutritional composition further comprises:
(f) sodium in an amount of about 50 mg/serving or more, about 60 mg/serving or more, about 70 mg/serving or more, about 80 mg/serving or more, about 90 mg/serving or more, or about 100 mg/serving or more;
(g) phosphorus in an amount of about 70 mg/serving or more, about 80 mg/serving or more, about 90 mg/serving or more, about 100 mg/serving or more, about 110 mg/serving or more, about 120 mg/serving or more, about 130 mg/serving or more, about 140 mg/serving or more, about 150 mg/serving or more;
(h) copper in an amount of about 0.05 mg/serving or more, about 0.10 mg/serving or more, about 0.15 mg/serving or more, about 0.20 mg/serving or more, about 0.25 mg/serving or more, or about 0.30 mg/serving or more;
(i) manganese in an amount of about 300 μg/serving or more, about 400 μg/serving or more, about 500 μg/serving or more, about 600 μg/serving or more;
(j) iodine in an amount of about 10 μg/serving or more, about 12 μg/serving or more, about 14 μg/serving or more, about 16 μg/serving or more, about 18 μg/serving or more, about 20 μg/serving or more, or about 22 μg/serving or more;
(k) chromium in an amount of about 3 μg/serving or more, about 4 μg/serving or more, about 5 μg/serving or more, about 6 μg/serving or more, or about 7 μg/serving or more; and/or
(l) molybdenum in an amount of about 3 μg/serving or more, about 4 μg/serving or more, about 5 μg/serving or more, about 6 μg/serving or more, or about 7 μg/serving or more.

In some embodiments, the nutritional composition comprises: (a) potassium in an amount of about 200 mg/100 kcal or more; (b) magnesium in an amount of about 30 mg/100 kcal or more; (c) iron in an amount of about 4 mg/100 kcal or more; (d) zinc in an amount of about 2 mg/100 kcal or more; and/or (e) selenium in an amount of about 5 μg/100 kcal or more.

In some embodiments, the nutritional composition further comprises: (f) sodium in an amount of about 50 mg/100 kcal or more; (g) phosphorus in an amount of about 70 mg/100 kcal or more; (h) copper in an amount of about 0.10 mg/100 kcal or more; (i) manganese in an amount of about 250 μg/100 kcal or more; (j) iodine in an amount of about 10 μg/100 kcal or more; (k) chromium in an amount of about 3 μg/100 kcal or more; and/or (l) molybdenum in an amount of about 3 μg/100 kcal or more.

In some embodiments, the nutritional composition further comprises antioxidants.

In some embodiments, the nutritional composition further comprises fiber.

### Protein, Carbohydrate, and Lipids

The nutritional composition may include all the nutrients required to maintain health. The nutritional composition may contain a protein source, a carbohydrate source and/or a lipid source.

The protein may be present in the nutritional composition in any suitable amount. For example, the protein content of the nutritional composition may be from about 30 g/L to about 200 g/L, from about 40 g/L to about 150 g/L, or from about 50 g/L to about 100 g/L, for example following reconstitution in water or milk.

In some embodiments, the nutritional composition comprises protein in an amount of from about 8 g/serving to about 14 g/serving, from about 10 g/serving to about 12 g/serving, or about 11 g/serving.

In some embodiments, the nutritional composition comprises protein in an amount of from about 3 g/100 kcal to about 7 g/100 kcal, from about 4 g/100 kcal to about 6 g/100 kcal, or about 5.1 g/100 kcal.

The protein source may be any protein source which is suitable for use in a nutritional composition. Protein sources based on, for example, milk proteins, whey, casein and mixtures thereof may be used, as well as protein sources based on soy. As far as whey proteins are concerned, the protein source may be based on acid whey or sweet whey or mixtures thereof.

The proteins may be intact or hydrolysed or a mixture of intact and hydrolysed proteins. By the term “intact” is meant that the main part of the proteins are intact, i.e. the molecular structure is not altered, for example at least 80%, at least 85%, at least 90%, at least 95%, at least 98%, or 100% of the proteins are not altered.

The term “hydrolysed” means in the context of the present invention a protein which has been hydrolysed or broken down into its component amino acids. The proteins may be either fully or partially hydrolysed. The degree of hydrolysis (DH) of the protein can be between 2 and 20, or between 8 and 40, or between 20 and 60 or between 20 and 80 or more than 10, 20, 40, 60, 80 or 90. Suitably, at least 70%, at least 80%, at least 85%, at least 90%, at least 95%, at least 98%, or 100% of the proteins may be hydrolysed. If hydrolysed proteins are required, the hydrolysis process may be carried out as desired and as is known in the art. For example, whey protein hydrolysates may be prepared by enzymatically hydrolysing the whey fraction in one or more steps.

The lipids (fat) may be present in the nutritional composition in any suitable amount. For example, the fat content of the nutritional composition may be from about 10 g/L to about 200 g/L, from about 15 g/L to about 150 g/L, or from about 20 g/L to about 100 g/L, for example following reconstitution in water or milk.

In some embodiments, the nutritional composition comprises fat in an amount of from about 5 g/serving to about 10 g/serving, from about 6 g/serving to about 9 g/serving, from about 7 g/serving to about 8 g/serving, or about 7.5 g/serving.

In some embodiments, the nutritional composition comprises fat in an amount of from about 2.0 g/100 kcal to about 5.0 g/100 kcal, from about 2.5 g/100 kcal to about 4.5 g/100 kcal, from about 3.0 g/100 kcal to about 4.0 g/100 kcal, or about 3.5 g/100 kcal.

Example fats for use in the nutritional composition of the invention include sunflower oil, low erucic acid rapeseed oil, safflower oil, canola oil, olive oil, coconut oil, palm kernel oil, soybean oil, fish oil, palm oleic, high oleic sunflower oil and high oleic safflower oil, and microbial fermentation oil containing long chain, polyunsaturated fatty acids. The fat may also be in the form of fractions derived from these oils, such as palm olein, medium chain triglycerides (MCT) and esters of fatty acids such as arachidonic acid, linoleic acid, palmitic acid, stearic acid, docosahexaeonic acid, linolenic acid, oleic acid, lauric acid, capric acid, caprylic acid, caproic acid, and the like. Further example fats include structured lipids (i.e. lipids that are modified chemically or enzymatically in order to change their structure).

Long chain polyunsaturated fatty acids, such as dihomo-γ-linolenic acid, arachidonic acid (ARA), eicosapentaenoic acid and docosahexaenoic acid (DHA), may also be added. The essential fatty acids linoleic and α-linolenic acid may also be added, as well small amounts of oils containing high quantities of preformed arachidonic acid and docosahexaenoic acid such as fish oils or microbial oils.

The carbohydrate may be present in the nutritional composition in any suitable amount. For example, the carbohydrate content of the nutritional composition may be from about 30 g/L to about 300 g/L, from about 40 g/L to about 250 g/L, or from about 50 g/L to about 200 g/L, for example following reconstitution in water or milk.

In some embodiments, the nutritional composition comprises carbohydrate in an amount of from about 20 g/serving to about 35 g/serving, from about 25 g/serving to about 30 g/serving, or about 27 g/serving.

In some embodiments, the nutritional composition comprises carbohydrate in an amount of from about 8 g/100 kcal to about 16 g/100 kcal, from about 9 g/100 kcal to about 15 g/100 kcal, from about 10 g/100 kcal to about 14 g/100 kcal, from about 11 g/100 kcal to about 13 g/100 kcal, from about 12 g/100 kcal to about 13 g/100 kcal, or about 12.7 g/100 kcal.

The carbohydrate source may be any carbohydrate source which is suitable for use in a nutritional composition. Some suitable carbohydrate sources include lactose, sucrose, saccharose, maltodextrin, starch and mixtures thereof.

### Other Components

The nutritional composition of the invention may contain emulsifiers and stabilisers such as soy, lecithin, citric acid esters of mono- and diglycerides, and the like.

The nutritional composition of the invention may also contain one or more carotenoid.

The nutritional composition of the invention may also contain other substances which may have a beneficial effect such as lactoferrin, osteopontin, TGFbeta, sIgA, glutamine, nucleotides, nucleosides, and the like.

The nutritional composition of the invention can further comprise at least one non-digestible oligosaccharide (e.g. prebiotics). Examples of prebiotics may be fructooligosaccharides and galactooligosaccharides.

The nutritional composition of the present invention can further comprise at least one probiotic. The term “probiotic” may refer to microbial cell preparations or components of microbial cells with beneficial effects on the health or well-being of the host. In particular, probiotics may improve gut barrier function. Examples of probiotic micro-organisms for use in the nutritional composition of the present invention include yeasts, such as Saccharomyces, Debaromyces, Candida, Pichia and Torulopsis; and bacteria, such as the genera Bifidobacterium, Bacteroides, Clostridium, Fusobacterium, Melissococcus, Propionibacterium, Streptococcus, Enterococcus, Lactococcus, Staphylococcus, Peptostrepococcus, Bacillus, Pediococcus, Micrococcus, Leuconostoc, Weissella, Aerococcus, Oenococcus and Lactobacillus. Specific examples of suitable probiotic microorganisms are: Saccharomyces cereviseae, Bacillus coagulans, Bacillus licheniformis, Bacillus subtilis, Bifidobacterium bifidum, Bifidobacterium infantis, Bifidobacterium longum, Enterococcus faecium, Enterococcus faecalis, Lactobacillus acidophilus, Lactobacillus alimentarius, Lactobacillus casei subsp. casei, Lactobacillus casei Shirota, Lactobacillus curvatus, Lactobacillus delbruckii subsp. lactis, Lactobacillus farciminus, Lactobacillus gasseri, Lactobacillus helveticus, Lactobacillus johnsonii, Lactobacillus rhamnosus (Lactobacillus GG), Lactobacillus sake, Lactococcus lactis, Micrococcus varians, Pediococcus acidilactici, Pediococcus pentosaceus, Pediococcus acidilactici, Pediococcus halophilus, Streptococcus faecalis, Streptococcus thermophilus, Staphylococcus carnosus and Staphylococcus xylosus.

### Energy

The nutritional composition may provide a suitable amount of energy to maintain health.

Suitably, the nutritional composition may comprise from about 500 kcal/L to about 3000 kcal/L, from about 750 kcal/L to about 2500 kcal/L, or from about 1000 kcal/L to about 2000 kcal/L, for example following reconstitution in water or milk.

In some embodiments, the nutritional composition comprises from about 100 kcal/serving to about 500 kcal/serving, from about 150 kcal/serving to about 400 kcal/serving, from about 200 kcal/serving to about 300 kcal/serving, or from about 200 kcal/serving to about 250 kcal/serving. In some embodiments, the nutritional composition comprises about 214 kcal/serving.

In some embodiments, the nutritional composition comprises from about 200 kcal/100 g to about 600 kcal/100 g, from about 300 kcal/100 g to about 500 kcal/100 g, or from about 350 kcal/100 g to about 450 kcal/100 g. In some embodiments, the nutritional composition comprises about 429 kcal/100 g.

### Supplements and Fortifiers

In one aspect, the present invention provides a supplement or fortifier comprising L-arginine, L-citrulline, a vitamin mixture, and a mineral mixture.

A “supplement” or “dietary supplement” may be used to complement the nutrition of an individual (it is typically used as such but it might also be added to a nutritional composition). The term “fortifier” may refer to liquid or solid compositions suitable for mixing with nutritional compositions.

The supplement or fortifier according to the invention may a supplement or fortifier for dietary management of a subject with disease related malnutrition. In particular, wherein the disease is an infection such as TB.

The supplement or fortifier may be in the form of for example sprinkles, sticks, tablets, capsules, pastilles, liquid, gel or spoonable food. In preferred embodiments, the supplement or fortifier is in the form of a powder supplement for sprinkling on food or in a beverage. A powder supplement may be provided in a sachet.

The supplement or fortifier may contain an organic or inorganic carrier material suitable for oral or enteral administration (e.g. maltodextrin) as well as vitamins, minerals trace elements and other micronutrients in accordance with the recommendations of Government bodies such as the USRDA. The supplement or fortifier may be provided in the form of unit doses.

Suitably, in the context of a supplement or fortifier, a “serving” may be from about 1 g to about 10 g. In some embodiments, a “serving” in the context of a supplement or fortifier is about 5 g.

### L-Arginine

The supplement or fortifier may comprise L-arginine in any therapeutically effective amount.

Suitably, the supplement or fortifier comprises L-arginine in an amount of about 0.5 g/serving or more and/or about 10.0 g/serving or less. In some embodiments, the supplement or fortifier comprises L-arginine in an amount of about 0.5 g/serving or more. In some embodiments, the supplement or fortifier comprises L-arginine in an amount of about 0.5 g/serving.

Suitably, the supplement or fortifier comprises L-arginine in an amount of about 1.0 wt % or more and/or about 20.0 wt % or less. In some embodiments, the supplement or fortifier comprises L-arginine in an amount of about 10.0 wt % or more. In some embodiments, the supplement or fortifier comprises L-arginine in an amount of about 10.0 wt %. This may be on a dry weight basis.

Suitably, the supplement or fortifier comprises L-arginine in an amount of about 0.5 g/100 kcal or more and/or about 10.0 g/100 kcal or less. In some embodiments, the supplement or fortifier comprises L-arginine in an amount of about 3.6 g/100 kcal or more. In some embodiments, the supplement or fortifier comprises L-arginine in an amount of about 3.6 g/100 kcal.

### L-Citrulline

The supplement or fortifier may comprise L-citrulline in any therapeutically effective amount.

Suitably, the supplement or fortifier comprises L-citrulline in an amount of about 0.5 g/serving or more and/or about 10.0 g/serving or less. In some embodiments, the supplement or fortifier comprises L-citrulline in an amount of about 0.5 g/serving or more. In some embodiments, the supplement or fortifier comprises L-citrulline in an amount of about 0.5 g/serving.

Suitably, the supplement or fortifier comprises L-citrulline in an amount of about 1.0 wt % or more and/or about 20.0 wt % or less. In some embodiments, the supplement or fortifier comprises L-citrulline in an amount of about 10.0 wt % or more. In some embodiments, the supplement or fortifier comprises L-citrulline in an amount of about 10.0 wt %. This may be on a dry weight basis.

Suitably, the supplement or fortifier comprises L-citrulline in an amount of about 0.5 g/100 kcal or more and/or about 10.0 g/100 kcal or less. In some embodiments, the supplement or fortifier comprises L-citrulline in an amount of about 3.6 g/100 kcal or more. In some embodiments, the supplement or fortifier comprises L-citrulline in an amount of about 3.6 g/100 kcal.

### Combination of L-Arginine and L-Citrulline

In some embodiments, the supplement or fortifier comprises: (a) L-arginine in an amount of about 0.5 g/serving or more; and (b) L-citrulline in an amount of about 0.5 g/serving or more.

In some embodiments, the supplement or fortifier comprises: (a) L-arginine in an amount of about 0.5 g/serving; and (b) L-citrulline in an amount of about 0.5 g/serving.

In some embodiments, the supplement or fortifier comprises: (a) L-arginine in an amount of about 10.0 wt % or more; and (b) L-citrulline in an amount of about 10.0 wt % or more, for example on a dry weight basis.

In some embodiments, the supplement or fortifier comprises: (a) L-arginine in an amount of about 10.0 wt %; and (b) L-citrulline in an amount of about 10.0 wt %, for example on a dry weight basis.

In some embodiments, the supplement or fortifier comprises: (a) L-arginine in an amount of about 3 g/100 kcal or more; and (b) L-citrulline in an amount of about 3 g/100 kcal or more.

In some embodiments, the supplement or fortifier comprises: (a) L-arginine in an amount of about 3.6 g/100 kcal; and (b) L-citrulline in an amount of about 3.6 g/100 kcal.

### Vitamin Mixture

The supplement or fortifier may comprise the vitamins in any therapeutically effective amount. Suitably, the supplement or fortifier comprises:
(a) vitamin A in an amount of about 200 μgRE/serving or more, about 250 μgRE/serving or more, about 300 μgRE/serving or more, about 350 μgRE/serving or more, or about 400 μgRE/serving or more;
(b) vitamin D2 in an amount of about 3 μg/serving or more, about 4 μg/serving or more, about 5 μg/serving or more, or about 6 μg/serving or more;
(c) vitamin E in an amount of about 2.0 mgTE/serving or more, about 2.5 mgTE/serving or more, about 3.0 mgTE/serving or more, about 3.5 mgTE/serving or more, or about 4.0 mgTE/serving or more;
(d) vitamin C in an amount of about 16 mg/serving or more, about 20 mg/serving or more, about 24 mg/serving or more, about 28 mg/serving or more, or about 32 mg/serving or more;
(e) vitamin B1 in an amount of about 0.30 mg/serving or more, about 0.35 mg/serving or more, about 0.40 mg/serving or more, about 0.45 mg/serving or more, about 0.50 mg/serving or more, or about 0.55 mg/serving or more;
(f) vitamin B2 in an amount of about 0.40 mg/serving or more, about 0.45 mg/serving or more, about 0.50 mg/serving or more, about 0.55 mg/serving or more, about 0.60 mg/serving or more, about 0.65 mg/serving or more, about 0.70 mg/serving or more, about 0.75 mg/serving or more, or about 0.80 mg/serving or more;
(g) niacin in an amount of about 3.0 mg/serving or more, about 3.5 mg/serving or more, about 4.0 mg/serving or more, about 4.5 mg/serving or more, about 5.0 mg/serving or more, or about 5.5 mg/serving or more;
(h) vitamin B6 in an amount of about 0.40 mg/serving or more, about 0.45 mg/serving or more, about 0.50 mg/serving or more, about 0.55 mg/serving or more, about 0.60 mg/serving or more, about 0.65 mg/serving or more, about 0.70 mg/serving or more, or about 0.75 mg/serving or more;
(i) folic acid in an amount of about 36 μg/serving or more, about 40 μg/serving or more, about 45 μg/serving or more, about 50 μg/serving or more, about 55 μg/serving or more, about 60 μg/serving or more, about 65 μg/serving or more, or about 70 μg/serving or more; and/or
(j) vitamin B12 in an amount of about 0.4 μg/serving or more, about 0.5 μg/serving or more, about 0.6 μg/serving or more, about 0.7 μg/serving or more, about 0.8 μg/serving or more, or about 0.9 μg/serving or more.

In some embodiments, the supplement or fortifier comprises: (a) vitamin A in an amount of about 2500 μgRE/100 kcal or more; (b) vitamin D2 in an amount of about 40 μg/100 kcal or more; (c) vitamin E in an amount of about 25 mgTE/100 kcal or more; (d) vitamin C in an amount of about 200 mg/100 kcal or more; (e) vitamin B1 in an amount of about 4 mg/100 kcal or more; (f) vitamin B2 in an amount of about 5 mg/100 kcal or more; (g) niacin in an amount of about 40 mg/100 kcal or more; (h) vitamin B6 in an amount of about 5 mg/100 kcal or more; (i) folic acid in an amount of about 500 μg/100 kcal or more; and/or (j) vitamin B12 in an amount of about 6 μg/100 kcal or more.

In some embodiments, the supplement or fortifier does not comprise any other vitamins other than those described above.

### Mineral Mixture

The supplement or fortifier may comprise the minerals in any therapeutically effective amount. Suitably, the supplement or fortifier comprises:
(a) potassium in an amount of about 250 mg/serving or more, about 300 mg/serving or more, about 350 mg/serving or more, about 400 mg/serving or more, about 450 mg/serving or more, or about 500 mg/serving or more;
(b) magnesium in an amount of about 33 mg/serving or more, about 35 mg/serving or more, about 40 mg/serving or more, about 45 mg/serving or more, about 50 mg/serving or more, about 55 mg/serving or more, about 60 mg/serving or more, or about 65 mg/serving or more;
(c) iron in an amount of about 5 mg/serving or more, about 6 mg/serving or more, about 7 mg/serving or more, about 8 mg/serving or more, about 9 mg/serving or more, about 10 mg/serving or more, or about 11 mg/serving or more;
(d) zinc in an amount of about 3 mg/serving or more, about 4 mg/serving or more, about 5 mg/serving or more, about 6 mg/serving or more, or about 7 mg/serving or more; and/or
(e) selenium in an amount of about 5 μg/serving or more, about 6 μg/serving or more, about 8 μg/serving or more, about 10 μg/serving or more, about 12 μg/serving or more, about 14 μg/serving or more, or about 16 μg/serving or more.
In some embodiments, the supplement or fortifier comprises: (a) potassium in an amount of about 3000 mg/100 kcal or more; (b) magnesium in an amount of about 400 mg/100 kcal or more; (c) iron in an amount of about 80 mg/100 kcal or more; (d) zinc in an amount of about 40 mg/100 kcal or more; and/or (e) selenium in an amount of about 100 μg/100 kcal or more.

In some embodiments, the supplement or fortifier does not comprise any other minerals other than those described above.

### Other Components

The supplement or fortifier of the invention may contain any other suitable components, such as protein and/or carbohydrates.

In some embodiments, the supplement or fortifier comprises protein in an amount of about 0.5 g/serving or more, about 0.6 g/serving or more, about 0.7 g/serving or more, about 0.8 g/serving or more, or about 0.9 g/serving or more.

In some embodiments, the supplement or fortifier comprises carbohydrate in an amount of about 1.0 g/serving or more, about 1.5 g/serving or more, about 2.0 g/serving or more, or about 2.5 g/serving or more.

In some embodiments, the supplement or fortifier does not comprise fat.

In some embodiments, the supplement or fortifier comprises from about 5 kcal/serving to about 20 kcal/serving, from about 10 kcal/serving to about 15 kcal/serving, or about 13.8 kcal/serving.

### Use as a Medicament

In one aspect, the present invention provides a combination of L-arginine, L-citrulline, a vitamin mixture, and a mineral mixture for use as a medicament.

In another aspect, the present invention provides use of a combination of L-arginine, L-citrulline, a vitamin mixture, and a mineral mixture for the manufacture of a medicament.

In another aspect, the present invention provides a nutritional composition, supplement, or fortifier according to the present invention for use as a medicament.

In another aspect, the present invention provides use of a nutritional composition, supplement, or fortifier according to the present invention for the manufacture of a medicament.

The medicament may be a food for dietary management of a subject, for example a subject having or at risk of an infection and/or an immunodeficiency, in particular a subject having or at risk of TB. Suitably, the medicament may be administered in the form of a nutritional composition, supplement, or fortifier according to the present invention.

The medicament may be an adjunctive therapy, for example the treatment or prevention of infection. As used herein, an “adjunctive therapy” (also called an “adjunct therapy”) may refer to a treatment given in addition to the main treatment or “primary therapy”, in order to maximize its effectiveness. For example, the primary therapy may be an antimicrobial therapy. In some embodiments, the medicament is an adjunctive therapy for the treatment of tuberculosis (TB), optionally wherein the primary therapy is a TB drug regimen.

### Subject

The combination of L-arginine, L-citrulline, a vitamin mixture, and a mineral mixture (e.g. in the form of a nutritional composition, supplement, or fortifier) may be administered to any subject in need thereof. The subject may be a human subject.

The subject may be any age. For example, the subject may be a child or an adult. The term “child” may refer to a subject aged under 18 years. The term “adult” may refer to a subject aged 18 years or older. In some embodiments, the subject is an adult.

The subject may have or be at risk of an infection and/or an immunodeficiency.

In some embodiments, the subject has or is at risk of an infection. The infection may be a bacterial infection. The infection may be a respiratory tract infection. The infection may be a lung infection. In some embodiments, the infection is tuberculosis (TB), such as pulmonary TB.

In some embodiments, the subject has latent tuberculosis and the infection is in its latent state.

In some embodiments, the subject has or is at risk of an immunodeficiency. Immunodeficiency disorders result in partial or full impairment of the immune system, leaving the patient unable to effectively resolve infections or disease. The immunodeficiency may be a secondary immunodeficiency, for example resulting from immunosuppressive agents, malnutrition, aging, medication, environmental toxins, or diseases such as cancer or HIV infection (see e.g. Tuano, K. S., et al., 2021. Annals of Allergy, Asthma & Immunology, 127(6), pp. 617-626).

### Routes of Administration and Dosage

The combination of L-arginine, L-citrulline, a vitamin mixture, and a mineral mixture (e.g. in the form of a nutritional composition, supplement, or fortifier) may be administered by any suitable route, for example by oral, parenteral, or enteral administration.

In some embodiments, the combination of L-arginine, L-citrulline, a vitamin mixture, and a mineral mixture (e.g. in the form of a nutritional composition, supplement, or fortifier) are administered orally.

The combination of L-arginine, L-citrulline, a vitamin mixture, and a mineral mixture may be administered simultaneously, sequentially, or separately. In preferred embodiments, the combination is administered simultaneously.

The combination of L-arginine, L-citrulline, a vitamin mixture, and a mineral mixture (e.g. in the form of a nutritional composition, supplement, or fortifier) may be administered in any suitable dose.

Suitably, L-arginine is administered to the subject in an amount of about 0.5 g/day or more, for example in an amount of about 0.5 g/day, about 0.6 g/day, about 0.7 g/day, about 0.8 g/day, about 0.9 g/day, about 1.0 g/day, about 1.1 g/day, about 1.2 g/day, about 1.3 g/day, about 1.4 g/day, or about 1.5 g/day. In some embodiments, L-arginine is administered to the subject in an amount of about 0.5 g/day or more. In some embodiments, L-arginine is administered to the subject in an amount of about 1.0 g/day or more. Suitably, L-arginine is administered to the subject in an amount of about 10.0 g/day or less. In some embodiments, L-arginine is administered to the subject in an amount of about 0.5 g/day. In other embodiments, L-arginine is administered to the subject in an amount of about 1.0 g/day.

Suitably, L-citrulline is administered to the subject in an amount of about 0.5 g/day or more, for example in an amount of about 0.5 g/day, about 0.6 g/day, about 0.7 g/day, about 0.8 g/day, about 0.9 g/day, about 1.0 g/day, about 1.1 g/day, about 1.2 g/day, about 1.3 g/day, about 1.4 g/day, or about 1.5 g/day. In some embodiments, L-citrulline is administered to the subject in an amount of about 0.5 g/day or more. In some embodiments, L-citrulline is administered to the subject in an amount of about 1.0 g/day or more. Suitably, L-citrulline is administered to the subject in an amount of about 10.0 g/day or less. In some embodiments, L-citrulline is administered to the subject in an amount of about 0.5 g/day. In other embodiments, L-citrulline is administered to the subject in an amount of about 1.0 g/day.

Suitably, L-arginine and L-citrulline are administered to the subject in a weight ratio of from about 0.1:1 to about 10:1, from about 0.2:1 to about 5:1, from about 0.25:1 to about 4:1, from about 0.3:1 to about 3:1, from about 0.5:1 to about 2:1, or about 1:1. In some embodiments, L-arginine and L-citrulline are administered to the subject in a weight ratio of about 1:1.

In some embodiments, L-arginine is administered to the subject in an amount of about 0.5 g/day and L-citrulline is administered to the subject in an amount of about 0.5 g/day.

In some embodiments, L-arginine is administered to the subject in an amount of about 1.0 g/day and L-citrulline is administered to the subject in an amount of about 1.0 g/day.

Suitably, each vitamin in the vitamin mixture is administered to the subject in an amount of about 10% RDA/day or more, about 20% RDA/day or more, about 30% RDA/day or more, about 40% RDA/day or more, about 50% RDA/day or more, about 60% RDA/day or more, about 70% RDA/day or more, about 80% RDA/day or more. Suitably, each vitamin in the vitamin mixture is administered to the subject in an amount of about 100% RDA/day or less.

In some embodiments, each vitamin in the vitamin mixture is administered to the subject in an amount of about 50% RDA/day or more.

Suitably, each mineral in the mineral mixture is administered to the subject in an amount of about 10% RDA/day or more, about 20% RDA/day or more, about 30% RDA/day or more, about 40% RDA/day or more, about 50% RDA/day or more, about 60% RDA/day or more, about 70% RDA/day or more, about 80% RDA/day or more. Suitably, each mineral in the mineral mixture is administered to the subject in an amount of about 100% RDA/day or less.

In some embodiments, each mineral in the mineral mixture is administered to the subject in an amount of about 15% RDA/day or more.

### Methods of Enhancing a Subject's Immune Responses

The agents, combinations, and compositions of the present invention may be used to enhance a subject's immune responses.

In one aspect, the present invention provides a combination of L-arginine and L-citrulline for use in enhancing a subject's immune responses.

In another aspect, the present invention provides use of a combination of L-arginine and L-citrulline for the manufacture of a medicament for enhancing a subject's immune responses.

In another aspect, the present invention provides a method of enhancing a subject's immune responses comprising administering a combination of L-arginine and L-citrulline to the subject.

In one aspect, the present invention provides a combination of L-arginine, L-citrulline, a vitamin mixture, and a mineral mixture for use in enhancing a subject's immune responses.

In another aspect, the present invention provides use of a combination of L-arginine, L-citrulline, a vitamin mixture, and a mineral mixture for the manufacture of a medicament for enhancing a subject's immune responses.

In another aspect, the present invention provides a method of enhancing a subject's immune responses comprising administering a combination of L-arginine, L-citrulline, a vitamin mixture, and a mineral mixture to the subject.

In another aspect, the present invention provides a method of enhancing a subject's immune responses comprising administering a combination of L-arginine, L-citrulline, a vitamin mixture, a mineral mixture and antioxidant mixture to the subject.

In another aspect, the present invention provides a nutritional composition, supplement, or fortifier according to the present invention for use in enhancing a subject's immune responses.

In another aspect, the present invention provides use of a nutritional composition, supplement, or fortifier according to the present invention for the manufacture of a medicament for enhancing a subject's immune responses.

In another aspect, the present invention provides a method of enhancing a subject's immune responses comprising administering to said subject a nutritional composition, supplement, or fortifier according to the present invention to the subject.

The primary role of the immune system is to protect the individual against pathogenic organisms including bacteria, viruses, fungi and parasites. So that it can provide effective protection against the wide array of threatening organisms, the human immune system has evolved to include many different cell types, many communicating molecules and multiple functional responses. Nutrition is one of multiple factors that determines the immune response and good nutrition is important in supporting the immune response (see e.g. Calder, P. C., 2021. Nutrition & Diabetes, 11(1), p. 19).

The subject's immune responses may, for example, be enhanced by increasing macrophage activity against pathogens, such as bacteria (e.g. Mycobacterium tuberculosis). NO plays an important role in the host defense against intracellular pathogens. Many invading organisms compromise host macrophages by impairing host NOS activity resulting in reduced NO production. Decreased host NO production will, therefore, result in a more sustainable niche for host infection. It is posited therefore, that NO donors given to TB patients will be able to compensate for the lack of endogenous NO by the compromised macrophages (see e.g. Jamaati, H., et al., 2017. Frontiers in microbiology, 8, p. 2008).

Methods of Treating, Preventing, and/or Reduce the Occurrence of Infection

The agents, combinations, and compositions of the present invention may be used to treat, prevent, and/or reduce the occurrence of an infection in a subject. The agents, combinations, and compositions of the present invention may reduce the amount of medicament (e.g., antimicrobial therapy) required to treat an infection.

As used herein, “treating” an infection means that the subject has an infection, and the agent, combination, or composition lessens, reduces, or improves at least one symptom associated with the infection and/or slow downs, reduces, or eliminates the infection.

As used herein, “prevent” an infection means that the subject has not yet contracted an infection and/or is not showing any symptoms of infection, and the agent, combination, or composition prevents infection and/or prevents or at least one symptom associated with infection.

As used herein, “reduce the occurrence” of an infection means that the agent, combination, or composition reduces the likelihood of infection and/or at least one symptom associated with infection.

As used herein, “prevent” and “reduce the occurrence” of infection can also mean that the subject has a latent form of tuberculosis and prevents and/or reduces at least one symptom of tuberculosis infection.

In one aspect, the present invention provides L-citrulline for use in treating, preventing, and/or reducing the occurrence of infection in a subject.

In one aspect, the present invention provides L-citrulline to reduce intracellular load of Mtb in macrophages.

In one aspect, the present invention provides L-arginine to reduce intracellular load of Mtb in macrophages.

In another aspect, the present invention provides use of L-citrulline for the manufacture of a medicament for treating, preventing, and/or reducing the occurrence of infection in a subject.

In another aspect, the present invention provides a method of treating, preventing, and/or reducing the occurrence of infection in a subject comprising administering L-citrulline to the subject.

In another aspect, the present invention provides a combination of L-arginine and L-citrulline for use in treating, preventing, and/or reducing the occurrence of infection in a subject.

In another aspect, the present invention provides use of a combination of L-arginine and L-citrulline for the manufacture of a medicament for treating, preventing, and/or reducing the occurrence of infection in a subject.

In another aspect, the present invention provides a method of treating, preventing, and/or reducing the occurrence of infection in a subject comprising administering a combination of L-arginine and L-citrulline to the subject.

In another aspect, the present invention provides a combination of L-arginine, L-citrulline, a vitamin mixture, and a mineral mixture for use in treating, preventing, and/or reducing the occurrence of infection in a subject.

In another aspect, the present invention provides use of a combination of L-arginine, L-citrulline, a vitamin mixture, and a mineral mixture for the manufacture of a medicament for treating, preventing, and/or reducing the occurrence of infection in a subject.

In another aspect, the present invention provides a method of treating, preventing, and/or reducing the occurrence of infection in a subject comprising administering a combination of L-arginine, L-citrulline, a vitamin mixture, and a mineral mixture to the subject.

In another aspect, the present invention provides a nutritional composition, supplement, or fortifier according to the present invention for use in treating, preventing, and/or reducing the occurrence of infection in a subject.

In another aspect, the present invention provides use of a nutritional composition, supplement, or fortifier according to the present invention for the manufacture of a medicament for treating, preventing, and/or reducing the occurrence of infection in a subject.

In another aspect, the present invention provides a method of treating, preventing, and/or reducing the occurrence of infection in a subject comprising administering to said subject a nutritional composition, supplement, or fortifier according to the present invention to the subject.

As used herein “infection” refers to the invasion of a subject's body tissues by any pathogen, including viruses, bacteria, fungi, parasite and arthropods. Preferably, the infection is a bacterial infection.

The infection may be a respiratory infection. A “respiratory tract infection” may refer to an infection of parts of the body involved in breathing, such as the sinuses, throat, airways or lungs. A respiratory infection may be an upper respiratory tract infection (URTI) and/or a lower respiratory tract infection (LRTI). URTI may be characterized by the following clinical symptoms: nasal (running nose, stuffed nose, blowing nose, yellow secretion, bloody secretion, sneezing); and/or pharyngeal (scratchy throat, sore throat, hoarseness); with fever (>38° C.) and/or otitis. LRTI may be characterized by the following clinical symptoms: pneumonia (an episode of pneumonia may be defined as the illness which is reported as cough or difficult breathing); and/or bronchial (cough and/or secretion and/or yellow secretion).

In preferred embodiments, the infection is tuberculosis (TB). In other preferred embodiments, the infection is pulmonary TB.

The agents, combinations and compositions of the present invention may reduce or prevent one or more symptoms and/or one or more signs of infection in the subject. The signs and symptoms of an infection may depend on the type of infection. Some signs of infection affect the whole body generally, such as fatigue, loss of appetite, weight loss, fevers, night sweats, chills, aches and pains. Others are specific to individual body parts, such as skin rashes, coughing, or a runny nose. For example, symptoms of infection may include: (i) positive sputum culture; (ii) cough; (iii) chest pain; (iv) fever; (v) night sweating; (vi) haemoptysis; (vii) dyspnea; and (viii) GI symptoms of diarrhea or constipation.

The agents, combinations and compositions of the present invention may reduce or prevent one or more symptoms and/or one or more signs of TB in the subject. For example, the agents, combinations and compositions of the present invention may reduce the subject's TBscore. The TBscore may consist of five symptoms (cough, hemoptysis, dyspnea, chest pain, and night sweats) and six signs (pale inferior conjunctivae, pulse>90 per minute, positive finding at lung auscultation, temperature>37° C. [axillary], body mass index [BMI]<18/<16, and mid-upper-arm circumference [MUAC] <220 mm/<200 mm) (see Rudolf, F., 2014. health action, 7(1), p. 24303).

The agents, combinations and compositions of the present invention may reduce or prevent one or more, two or more, three or more, four or more, five or more, six or more, or seven or more symptoms of infection (e.g. TB) in the subject. In some embodiments, the symptoms of infection (e.g. TB) include: (i) positive sputum culture; (ii) cough; (iii) chest pain; (iv) fever; (v) night sweating; (vi) haemoptysis; (vii) dyspnea; and (viii) GI symptoms of diarrhea or constipation.

In some embodiments, the agents, combinations and compositions of the present invention may reduce positive sputum culture in a subject. Methods to process sputum cultures are known in the art (see e.g. Steingart, K. R., et al., 2006. The Lancet infectious diseases, 6(10), pp. 664-674).

In some embodiments, the agents, combinations and compositions of the present invention may reduce cough in a subject.

In some embodiments, the agents, combinations and compositions of the present invention may reduce chest pain in a subject.

In some embodiments, the agents, combinations and compositions of the present invention may reduce fever in a subject.

In some embodiments, the agents, combinations and compositions of the present invention may reduce night sweating in a subject.

In some embodiments, the agents, combinations and compositions of the present invention may reduce haemoptysis in a subject. Haemoptysis is the coughing of blood originating from the respiratory tract below the level of the larynx.

In some embodiments, the agents, combinations and compositions of the present invention may reduce dyspnea in a subject. Dyspnea is commonly referred to as shortness of breath.

In some embodiments, the agents, combinations and compositions of the present invention may reduce GI symptoms of diarrhea or constipation in a subject.

### Methods of Accelerating Wound Healing

The agents, combinations, and compositions of the present invention may be used to accelerating wound healing.

Wound healing, as a normal biological process in the human body, is achieved through four precisely and highly programmed phases: hemostasis, inflammation, proliferation, and remodelling (see e.g. Guo, S. A. and DiPietro, L. A., 2010. Journal of dental research, 89(3), pp. 219-229). Methods to assess the rate of wound healing are known in the art (see e.g. Barber, S., 2008. Ostomy Wound Management, 54(3), p. 42).

A role for dietary intervention in the form of arginine supplementation has been proposed to normalize or enhance wound healing in humans (see e.g. Stechmiller, J. K., et al., 2005. Nutrition in Clinical Practice, 20(1), pp. 52-61).

In one aspect, the present invention provides a combination of L-arginine and L-citrulline for use in accelerating wound healing.

In another aspect, the present invention provides use of a combination of L-arginine and L-citrulline for the manufacture of a medicament for accelerating wound healing.

In another aspect, the present invention provides a method of accelerating wound healing comprising administering a combination of L-arginine and L-citrulline to a subject in need thereof.

In one aspect, the present invention provides a combination of L-arginine, L-citrulline, a vitamin mixture, and a mineral mixture for use in accelerating wound healing.

In another aspect, the present invention provides use of a combination of L-arginine, L-citrulline, a vitamin mixture, and a mineral mixture for the manufacture of a medicament for accelerating wound healing.

In another aspect, the present invention provides a method of accelerating wound healing comprising administering a combination of L-arginine, L-citrulline, a vitamin mixture, and a mineral mixture to a subject in need thereof.

In another aspect, the present invention provides a nutritional composition, supplement, or fortifier according to the present invention for use in accelerating wound healing.

In another aspect, the present invention provides use of a nutritional composition, supplement, or fortifier according to the present invention for the manufacture of a medicament for accelerating wound healing.

In another aspect, the present invention provides a method of accelerating wound healing comprising administering to said subject a nutritional composition, supplement, or fortifier according to the present invention to a subject in need thereof.

Methods of Treating and/or Preventing Chronic Obstructive Pulmonary Disease (COPD)

The agents, combinations, and compositions of the present invention may be used to treat and/or prevent chronic obstructive pulmonary disease (COPD).

Chronic obstructive pulmonary disease (COPD) is a type of progressive lung disease characterized by long-term respiratory symptoms and airflow limitation. Methods to diagnose and monitor COPD are known in the art (see e.g. Johns, D. P., et al., 2014. Journal of thoracic disease, 6(11), p. 1557).

The production of NO in the lung and airways can play a number of roles during lung development, regulates airway and vascular smooth muscle tone, and is involved in inflammatory processes and host defense (see e.g. Scott, J. A., et al., 2021. Frontiers in Pharmacology, 12, p. 627503).

In one aspect, the present invention provides a combination of L-arginine and L-citrulline for use in treating and/or preventing COPD.

In another aspect, the present invention provides use of a combination of L-arginine and L-citrulline for the manufacture of a medicament for treating and/or preventing COPD.

In another aspect, the present invention provides a method of treating and/or preventing COPD comprising administering a combination of L-arginine and L-citrulline to a subject in need thereof.

In one aspect, the present invention provides a combination of L-arginine, L-citrulline, a vitamin mixture, and a mineral mixture for use in treating and/or preventing COPD.

In another aspect, the present invention provides use of a combination of L-arginine, L-citrulline, a vitamin mixture, and a mineral mixture for the manufacture of a medicament for treating and/or preventing COPD.

In another aspect, the present invention provides a method of treating and/or preventing COPD comprising administering a combination of L-arginine, L-citrulline, a vitamin mixture, and a mineral mixture to a subject in need thereof.

In another aspect, the present invention provides a nutritional composition, supplement, or fortifier according to the present invention for use in treating and/or preventing COPD.

In another aspect, the present invention provides use of a nutritional composition, supplement, or fortifier according to the present invention for the manufacture of a medicament for treating and/or preventing COPD.

In another aspect, the present invention provides a method of treating and/or preventing COPD comprising administering to said subject a nutritional composition, supplement, or fortifier according to the present invention to a subject in need thereof.

### Methods of Manufacture

In one aspect, the present invention provides a method of producing a nutritional composition, supplement, or fortifier according to the present invention.

The method may comprise: (i) providing a base nutritional composition, supplement, or fortifier; and (ii) adding L-arginine, L-citrulline, a vitamin mixture, and a mineral mixture to the base nutritional composition, supplement, or fortifier to provide a nutritional composition, supplement, or fortifier according to the present invention.

The nutritional composition, supplement, or fortifier of the present invention may be prepared by any suitable method known in the art.

For example, a nutritional composition, supplement, or fortifier may be prepared by blending together a protein source, a carbohydrate source and optionally a fat source in appropriate proportions. If used, emulsifiers may be included at this point. Vitamins and minerals may be added at this point but they may be added later to avoid thermal degradation. Any lipophilic vitamins, emulsifiers and the like may be dissolved into the fat source prior to blending. Water, preferably water which has been subjected to reverse osmosis, may then be mixed in to form a liquid mixture. Commercially available liquefiers may be used to form the liquid mixture.

L-arginine and L-citrulline may be added at this point, especially if the final product is to have a liquid form. If the final product is to be a powder, they may likewise be added at this stage if desired. Alternatively the L-arginine and L-citrulline may be added later to avoid thermal degradation.

The liquid mixture may then be homogenised, for example in two stages.

The liquid mixture may then be thermally treated to reduce bacterial loads, by rapidly heating the liquid mixture to a temperature in the range between about 80° C. and about 150° C. for a duration between about 5 seconds and about 5 minutes, for example. This may be carried out by means of steam injection, an autoclave or a heat exchanger, for example a plate heat exchanger.

Then, the liquid mixture may be cooled to between about 60° C. and about 85° C. for example by flash cooling. The liquid mixture may then be again homogenised, for example in two stages between about 10 MPa and about 30 MPa in the first stage and between about 2 MPa and about 10 MPa in the second stage. The homogenised mixture may then be further cooled to add any heat sensitive components, such as vitamins and minerals. The pH and solids content of the homogenised mixture are conveniently adjusted at this point.

If a liquid composition is preferred, the homogenised mixture may be sterilised then aseptically filled into suitable containers or may be first filled into the containers and then retorted.

If the final product is to be a powder, the homogenised mixture may be transferred to a suitable drying apparatus such as a spray dryer or freeze dryer and converted to powder. The powder may have a moisture content of less than about 5% by weight. The L-arginine, L-citrulline, vitamins and minerals may also or alternatively be added at this stage by dry-mixing or by blending.

### EXAMPLES

The invention will now be further described by way of examples, which are meant to serve to assist one of ordinary skill in the art in carrying out the invention and are not intended in any way to limit the scope of the invention.

### Example 1—Example Nutritional Composition

An example powder nutritional composition according to the present invention is shown below. The example powder nutritional composition may be reconstituted with water for dietary management of TB.

| | per 100 g | | per serving | | per 100 kcal | |
|---|---|---|---|---|---|---|
| Energy | 428.6 | kcal | 214.3 | kcal | 100 | kcal |
| Protein | 22 | g | 11 | g | 5.13 | g |
| Fat | 15 | g | 7.5 | g | 3.50 | g |
| Carbohydrates | 54.4 | g | 27.2 | g | 12.69 | g |
| L-Arginine | 2 | g | 1 | g | 0.47 | g |
| L-Citrulline | 2.5 | g | 1.25 | g | 0.58 | g |
| Vitamin A | 1276 | μgRE | 638 | μgRE | 297.71 | μgRE |
| Vitamin D2 | 19.5 | μgD | 9.75 | μgD | 4.55 | μgD |
| Vitamin E | 12.9 | mgTE | 6.45 | mgTE | 3.01 | mgTE |
| Vitamin K1 | 24 | μg | 12 | μg | 5.60 | μg |
| Vitamin C | 102 | mg | 51 | mg | 23.80 | mg |
| Vitamin B1 | 1.92 | mg | 0.96 | mg | 0.45 | mg |
| Vitamin B2 | 2.8 | mg | 1.4 | mg | 0.65 | mg |
| Niacin | 19 | mg | 9.5 | mg | 4.43 | mg |
| Pantothenic | 1.5 | mg | 0.75 | mg | 0.35 | mg |
| Acid | | | | | | |
| Vitamin B6 | 2.5 | mg | 1.25 | mg | 0.58 | mg |
| Folic acid | 240 | μg | 120 | μg | 56.00 | μg |
| Vitamin B12 | 2.9 | μg | 1.45 | μg | 0.68 | μg |
| Biotin | 9 | μg | 4.5 | μg | 2.10 | μg |
| Sodium | 212 | mg | 106 | mg | 49.46 | mg |
| Chloride | 355 | mg | 177.5 | mg | 82.83 | mg |
| Potassium | 1050 | mg | 525 | mg | 244.98 | mg |
| Calcium | 300 | mg | 150 | mg | 70.00 | mg |
| Phosphorus | 300 | mg | 150 | mg | 70.00 | mg |
| Magnesium | 132 | mg | 66 | mg | 30.80 | mg |
| Iron | 17.4 | mg | 8.7 | mg | 4.06 | mg |
| Zinc | 10.2 | mg | 5.1 | mg | 2.38 | mg |
| Copper | 0.6 | mg | 0.3 | mg | 0.14 | mg |
| Manganese | 1200 | μg | 600 | μg | 279.98 | μg |
| Iodine | 45 | μg | 22.5 | μg | 10.50 | μg |
| Selenium | 24 | μg | 12 | μg | 5.60 | μg |
| Chromium | 15 | μg | 7.5 | μg | 3.50 | μg |
| Molybdenum | 13.5 | μg | 6.75 | μg | 3.15 | μg |

### Example 2—Example Nutritional Supplement

An example powder nutritional supplement according to the present invention is shown below. The example powder nutritional supplement may be sprinkled on food for dietary management of TB.

| | per 100 g | | per serving | |
|---|---|---|---|---|
| Energy | 276 | kcal | 13.8 | kcal |
| Protein | 18.2 | g | 0.91 | g |
| Carbohydrates | 50.8 | g | 2.54 | g |
| L-Arginine | 10 | g | 0.5 | g |
| L-Citrulline | 10 | g | 0.5 | g |
| Vitamin A | 8000 | μgRE | 400 | μgRE |
| Vitamin D2 | 120 | μg | 6 | μg |
| Vitamin E | 80 | mgTE | 4 | mgTE |
| Vitamin C | 640 | mg | 32 | mg |
| Vitamin B1 | 11.2 | mg | 0.56 | mg |
| Vitamin B2 | 16 | mg | 0.8 | mg |
| Niacin | 112 | mg | 5.6 | mg |
| Vitamin B6 | 15.2 | mg | 0.76 | mg |
| Folic acid | 1440 | μg | 72 | μg |
| Vitamin B12 | 17.6 | μg | 0.88 | μg |
| Potassium | 10500 | mg | 525 | mg |
| Magnesium | 1320 | mg | 66 | mg |
| Iron | 232 | mg | 11.6 | mg |
| Zinc | 136 | mg | 6.8 | mg |
| Selenium | 320 | μg | 16 | μg |

### Example 3—Effect of L-Arginine and L-Citrulline on Intracellular Mtb Load in Macrophages

A critical step of tuberculosis (TB) infection is uptake of the foreign Mtb into macrophages, in particular, the macrophages of lung tissue where Mtb may survive for extended periods of time as macrophages recognize bacteria by their surface proteins and glycolipids.

Mycobacterium tuberculosis (Mtb), has developed mechanisms to evade host defense responses and persist within host cells such as macrophages for prolonged periods of time.

Therefore, we tested the effect of L-Arginine and L-Citrulline treatment in vitro on intracellular Mtb growth in THP-1 macrophages and compared to no treatment to see whether there may be a direct effect on macrophages.

THP-1 cells were differentiated into macrophages and seeded into 24 well plates at 200,000 cells per well in DMEM media containing 10% FBS without Arginine.

L-Arg or L-Cit were added to the macrophages either 24 hrs prior to infection or concurrently with infection or 24 hrs after infection with Mtb at a multiplicity of infection (MOI) of 1.0. Uninfected and untreated cells were included as controls.

The infection was allowed up to 96 hours. At every 24 hrs, starting from T=0 until 96 hours, samples were procured. The samples include cell-free supernatant, whole cells and cell lysates.

We have administered L-Arginine in doses of 100 and 1000 UM and followed the effect on post-infection with Mtb for up to 120 hours. We have been able to observe significant reductions in Mtb load with treatment using Arginine in macrophages compared to no treatment.

We have administered L-Citrulline in doses of 100 and 1000 UM and followed the effect on post-infection with Mtb for up to 120 hours. We have been able to observe significant reductions in Mtb load with treatment using Citrulline in macrophages compared to no treatment.

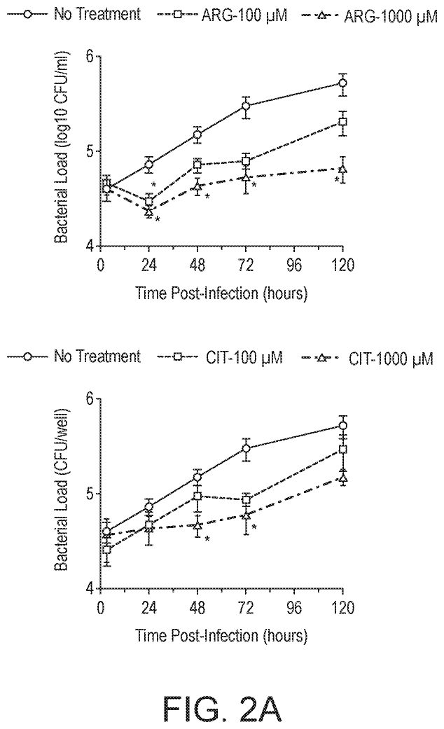

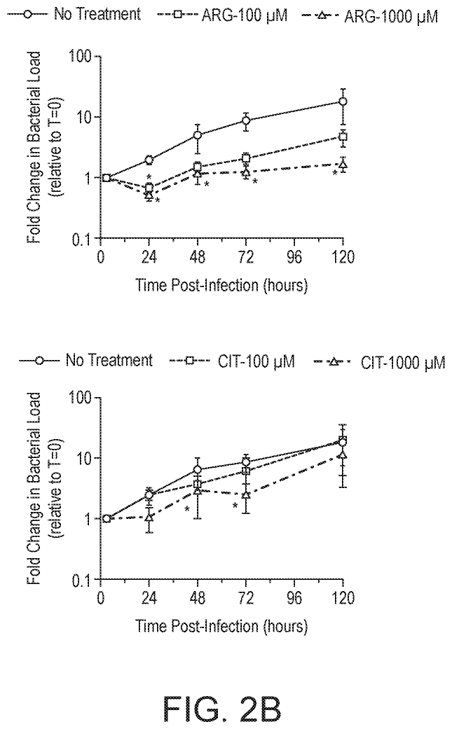

As shown in FIGS. 2A and 2B, we have been able to demonstrate that compounds of the invention help to reduce of MTb load in macrophages. This is an important step in the managing tuberculosis by preventing, and/or reducing the occurrence of infection, and reducing the occurrence or reoccurrence of one or more symptoms of infection of TB.

### EMBODIMENTS

Various preferred features and embodiments of the present invention will now be described with reference to the following numbered paragraphs (paras).
1. A nutritional composition, supplement, or fortifier comprising L-arginine, L-citrulline, a vitamin mixture, and a mineral mixture.
2. The nutritional composition, supplement, or fortifier according to para 1, wherein the L-arginine is present in an amount such that the nutritional composition, supplement, or fortifier provides L-arginine in a dosage of about 0.5 g/day or more.
3. The nutritional composition, supplement, or fortifier according to para 1 or 2, wherein the L-citrulline is present in an amount such that the nutritional composition, supplement, or fortifier provides L-citrulline in a dosage of about 0.5 g/day or more.
4. The nutritional composition, supplement, or fortifier according to any preceding para, wherein the L-arginine and L-citrulline are present in a weight ratio of from about 0.1:1 to about 10:1, from about 0.2:1 to about 5:1, from about 0.25:1 to about 4:1, from about 0.3:1 to about 3:1, from about 0.5:1 to about 2:1, or about 1:1.
5. The nutritional composition, supplement, or fortifier according to any preceding para, wherein the vitamin mixture comprises one or more of: vitamin A, vitamin D2, vitamin E, vitamin C, vitamin B1, vitamin B2, niacin, vitamin B6, folic acid, and vitamin B12.
6. The nutritional composition, supplement, or fortifier according to any preceding para, wherein the vitamin mixture comprises vitamin A, vitamin D2, vitamin E, vitamin C, vitamin B1, vitamin B2, niacin, vitamin B6, folic acid, and vitamin B12.
7. The nutritional composition, supplement, or fortifier according to para 6, wherein each of vitamin A, vitamin D2, vitamin E, vitamin C, vitamin B1, vitamin B2, niacin, vitamin B6, folic acid, and vitamin B12 is present in an amount such that the nutritional composition, supplement, or fortifier provides the vitamin in a dosage of about 50% RDA/day or more.
8. The nutritional composition, supplement, or fortifier according to any preceding para, wherein the mineral mixture comprises one or more of: potassium, magnesium, iron, zinc, and selenium.
9. The nutritional composition, supplement, or fortifier according to any preceding para, wherein the mineral mixture comprises potassium, magnesium, iron, zinc, and selenium.
10. The nutritional composition, supplement, or fortifier according to para 9, wherein each of potassium, magnesium, iron, zinc, and selenium are present in an amount such that the nutritional composition, supplement, or fortifier provides the mineral in a dosage of about 15% RDA/day or more.
11. The nutritional composition, supplement, or fortifier according to para 9 or 10, wherein each of iron, zinc, and selenium are present in an amount such that the nutritional composition, supplement, or fortifier provides the mineral in a dosage of about 30% RDA/day or more.
12. The nutritional composition, supplement, or fortifier according to any preceding para, wherein the nutritional composition, supplement, or fortifier further comprises carbohydrate and/or protein.
13. The nutritional composition, supplement, or fortifier according to any preceding para, wherein the nutritional composition, supplement, or fortifier is in the form of a powder.
14. The nutritional composition, supplement, or fortifier according to any preceding para, wherein the nutritional composition, supplement, or fortifier is a food for special dietary uses.
15. The nutritional composition, supplement, or fortifier according to para 14, wherein the food for special dietary uses is provided in the form of a powder for reconstitution with water or milk.
16. The nutritional composition, supplement, or fortifier according to para 14 or 15, wherein the food for special dietary uses is nutritionally complete.
17. The nutritional composition, supplement, or fortifier according to any of paras 14-16, wherein the food for special dietary uses comprises L-arginine in an amount of about 1 g/serving or more.
18. The nutritional composition, supplement, or fortifier according to any of paras 14-17, wherein the food for special dietary uses comprises L-citrulline in an amount of 1 g/serving or more.
19. The nutritional composition, supplement, or fortifier according to any of paras 14-18, wherein the food for special dietary uses comprises: (a) L-arginine in an amount of about 1 g/serving or more; and (b) L-citrulline in an amount of about 1 g/serving or more.
20. The nutritional composition, supplement, or fortifier according to any of paras 14-19, wherein the food for special dietary uses comprises: (a) vitamin A in an amount of about 300 μgRE/serving or more; (b) vitamin D2 in an amount of about 5 μg/serving or more; (c) vitamin E in an amount of about 3 mgTE/serving or more; (d) vitamin C in an amount of about 25 mg/serving or more; (e) vitamin B1 in an amount of about 0.5 mg/serving or more; (f) vitamin B2 in an amount of about 0.7 mg/serving or more; (g) niacin in an amount of about 5 mg/serving or more; (h) vitamin B6 in an amount of about 0.6 mg/serving or more; (i) folic acid in an amount of about 60 μg/serving or more; and (j) vitamin B12 in an amount of about 0.7 μg/serving or more.
21. The nutritional composition, supplement, or fortifier according to any of paras 14-20, wherein the food for special dietary uses comprises: (a) potassium in an amount of about 250 mg/serving or more; (b) magnesium in an amount of about 30 mg/serving or more; (c) iron in an amount of about 4 mg/serving or more; (d) zinc in an amount of about 2 mg/serving or more; and (e) selenium in an amount of about 6 μg/serving or more.
22. The nutritional composition, supplement, or fortifier according to any of paras 14-21, wherein the food for special dietary uses comprises: (a) protein in an amount of from about 8 g/serving to about 14 g/serving, from about 10 g/serving to about 12 g/serving, or about 11 g/serving; (b) fat in an amount of from about 5 g/serving to about 10 g/serving, from about 6 g/serving to about 9 g/serving, or about 7.5 g/serving; and (c) carbohydrate in an amount of from about 20 g/serving to about 35 g/serving, from about 25 g/serving to about 30 g/serving, or about 27 g/serving.
23. The nutritional composition, supplement, or fortifier according to any of paras 14-22, wherein the food for special dietary uses comprises from about 100 to about 300 kcal/serving, from about 150 to about 250 kcal/serving, or about 200 kcal/serving.
24. The nutritional composition, supplement, or fortifier according to any of paras 1-13, wherein the nutritional composition, supplement, or fortifier is a nutritional supplement.
25. The nutritional composition or supplement according to para 24, wherein the nutritional supplement is a powder supplement for sprinkling on food or in a beverage.
26. The nutritional composition, supplement, or fortifier according to para 24 or 25, wherein the nutritional supplement comprises L-arginine in an amount of about 0.5 g/serving or more.
27. The nutritional composition, supplement, or fortifier according to any of paras 24-26, wherein the nutritional supplement comprises L-citrulline in an amount of about 0.5 g/serving or more.
28. The nutritional composition, supplement, or fortifier according to any of paras 24-27, wherein the nutritional supplement comprises: (a) L-arginine in an amount of about 0.5 g/serving or more; and (b) L-citrulline in an amount of about 0.5 g/serving or more.
29. The nutritional composition, supplement, or fortifier according to any of paras 24-28, wherein the nutritional supplement comprises: (a) vitamin A in an amount of about 200 μgRE/serving or more; (b) vitamin D2 in an amount of about 3 μg/serving or more; (c) vitamin E in an amount of about 2 mgTE/serving or more; (d) vitamin C in an amount of about 16 mg/serving or more; (e) vitamin B1 in an amount of about 0.3 mg/serving or more; (f) vitamin B2 in an amount of about 0.4 mg/serving or more; (g) niacin in an amount of about 3 mg/serving or more; (h) vitamin B6 in an amount of about 0.4 mg/serving or more; (i) folic acid in an amount of about 36 μg/serving or more; and (j) vitamin B12 in an amount of about 0.4 μg/serving or more.
30. The nutritional composition, supplement, or fortifier according to any of paras 23-29, wherein the nutritional supplement comprises: (a) potassium in an amount of about 250 mg/serving or more; (b) magnesium in an amount of about 33 mg/serving or more; (c) iron in an amount of about 5 mg/serving or more; (d) zinc in an amount of about 3 mg/serving or more; and (e) selenium in an amount of about 5 μg/serving or more.
31. The nutritional composition, supplement, or fortifier according to any of paras 1-30, wherein the nutritional composition, supplement, or fortifier is a nutritional composition, supplement, or fortifier for dietary management of a subject with an infection and/or having an immunodeficiency, preferably wherein the nutritional composition, supplement, or fortifier is a nutritional composition, supplement, or fortifier for dietary management of a subject with tuberculosis (TB).
32. The nutritional composition, supplement, or fortifier according to any of paras 1-30, for use in enhancing immune responses.
33. The nutritional composition, supplement, or fortifier according to any of paras 1-30, for use in treating, preventing, and/or reducing the risk of occurrence of an infection, preferably wherein the infection is tuberculosis (TB), more preferably wherein the infection is pulmonary TB.
34. The nutritional composition, supplement, or fortifier for according to any of paras 1-30, for use as an adjunctive therapy in combination with an antimicrobial therapy.
35. The nutritional composition, supplement, or fortifier for according to any of paras 1-30, for use in treating, preventing, and/or reducing the risk of occurrence of one or more symptoms of infection, wherein the symptoms of infection are selected from: (i) positive sputum culture; (ii) cough; (iii) chest pain; (iv) fever; (v) night sweating; (vi) haemoptysis; and (vii) dyspnea and (viii) GI symptoms of diarrhea or constipation.
36. The nutritional composition, supplement, or fortifier according to any of paras 1-30, for use in accelerating wound healing.
37. The nutritional composition, supplement, or fortifier according to any of paras 1-30, for use in treating or preventing chronic obstructive pulmonary disease (COPD).
38. A method of enhancing a subject's immune responses, the method comprising administering to the subject a nutritional composition, supplement, or fortifier according to any of paras 1-30.
39. A method of treating, preventing, and/or reducing the risk of occurrence or reoccurrence of an infection in a subject, the method comprising administering to the subject a nutritional composition, supplement, or fortifier according to any of paras 1-30, preferably wherein the infection is tuberculosis (TB).
40. A method of treating, preventing, and/or reducing the risk of occurrence or reoccurrence of one or more symptoms of infection in a subject, the method comprising administering to the subject a nutritional composition, supplement, or fortifier according to any of paras 1-30, wherein the symptoms of infection are selected from: (i) positive sputum culture; (ii) cough; (iii) chest pain; (iv) fever; (v) night sweating; (vi) haemoptysis; (vii) dyspnea; and (viii) GI symptoms of diarrhea or constipation.
41. A method of accelerating wound healing in a subject, the method comprising administering to the subject a nutritional composition, supplement, or fortifier according to any of paras 1-30.
42. A method of treating or preventing chronic obstructive pulmonary disease (COPD) in a subject, the method comprising administering to the subject a nutritional composition, supplement, or fortifier according to any of paras 1-30.
43. L-citrulline for use in treating, preventing, and/or reducing the risk of occurrence of tuberculosis (TB) in a subject.
44. The L-citrulline for use according to para 43, wherein the L-citrulline is administered to the subject orally.
45. The L-citrulline for use according to para 43 or 44, wherein the L-citrulline is administered to the subject in an amount of about 0.5 g/day or more.
46. The L-citrulline for use according to any of paras 43-45, wherein the L-citrulline is administered to the subject in the form of a nutritional composition, supplement, or fortifier, optionally wherein the nutritional composition, supplement, or fortifier is defined according to any of paras 1-30.
47. The L-citrulline for use according to any of paras 43-46, wherein the L-citrulline is administered in combination with L-arginine.
48. The L-citrulline for use according to para 47, wherein the L-citrulline and L-arginine are administered simultaneously.
49. The L-citrulline for use according to para 47 or 48, wherein the L-arginine is administered in an amount of about 0.5 g/day or more.
50. The L-citrulline for use according to any of paras 43-49, wherein the subject is an adult human.
51. The L-citrulline for use according to any of paras 43-50, wherein the L-citrulline is administered to the subject as an adjunctive therapy, wherein the primary therapy is an antimicrobial therapy.
52. The L-citrulline for use according to any of paras 43-51, wherein one or more symptoms of infection are reduced or prevented in the subject, wherein the symptoms of infection are selected from: (i) positive sputum culture; (ii) cough; (iii) chest pain; (iv) fever; (v) night sweating; (vi) haemoptysis; (vii) dyspnea; and (viii) GI symptoms of diarrhea or constipation.
53. A method of treating or reducing the risk of tuberculosis (TB) in a subject, the method comprising administering to the subject a therapeutically effective amount of L-citrulline.
54. A method of reducing the occurrence of one or more symptoms of infection in a subject with tuberculosis (TB), comprising administering to the subject a therapeutically effective amount of L-citrulline, wherein the symptoms of infection are selected from: (i) positive sputum culture; (ii) cough; (iii) chest pain; (iv) fever (v) night sweating; (vi) haemoptysis; (vii) dyspnea; and GI symptoms of diarrhea or constipation.
55. A combination of L-arginine and L-citrulline for use in enhancing a subject's immune responses.
56. A combination of L-arginine and L-citrulline for use in treating, preventing, and/or reducing the risk of occurrence of an infection in a subject.
57. The combination for use according to para 56, wherein the infection is tuberculosis (TB).
58. The combination for use according to any of paras 55-57, wherein one or more symptoms of infection are reduced or prevented in the subject, wherein the symptoms of infection are selected from: (i) positive sputum culture; (ii) cough; (iii) chest pain; (iv) fever; (v) night sweating; (vi) haemoptysis; (vii) dyspnea and (viii) GI symptoms of diarrhea or constipation.
59. A combination of L-arginine and L-citrulline for use in accelerating wound healing in a subject.
60. A combination of L-arginine and L-citrulline for use in treating and/or preventing chronic obstructive pulmonary disease (COPD) in a subject.
61. The combination for use according to any of paras 55-60, wherein the L-citrulline is administered to the subject in an amount of about 0.5 g/day or more and/or wherein the L-arginine is administered to the subject in an amount of about 0.5 g/day or more.
62. The combination for use according to any of paras 55-61, wherein the L-citrulline and L-arginine are administered to the subject simultaneously.
63. The combination for use according to any of paras 55-62, wherein the combination is administered to the subject orally.
64. The combination for use according to any of paras 55-63, wherein the combination is administered to the subject in the form of a nutritional composition, supplement, or fortifier, optionally wherein the nutritional composition, supplement, or fortifier is defined according to any of paras 1 to 30.
65. The combination for use according to any of paras 55-64, wherein the subject is an adult human.
66. The combination for use according to any of paras 55-65, wherein the subject has or is at risk of an immunodeficiency.
67. The combination for use according to any of paras 55-77, wherein the combination is administered to the subject as an adjunctive therapy, wherein the primary therapy is an antimicrobial therapy.
68. A method of enhancing a subject's immune responses, the method comprising administering to the subject an effective amount of a combination of L-arginine and L-citrulline.
69. A method of treating or reducing the risk of infection in a subject, the method comprising administering to the subject an effective amount of a combination of L-arginine and L-citrulline.
70. The method according to para 69, wherein the infection is tuberculosis (TB).
71. A method of reducing the occurrence of one or more symptoms of infection in a subject, the method comprising administering to the subject an effective amount of a combination of L-arginine and L-citrulline, wherein the symptoms of infection are selected from: (i) positive sputum culture; (ii) cough; (iii) chest pain; (iv) fever; (v) night sweating; (vi) haemoptysis; and (vii) dyspnea; and (viii) GI symptoms of diarrhea or constipation.
72. A method of accelerating wound healing in a subject, the method comprising administering to the subject an effective amount of a combination of L-arginine and L-citrulline.
73. A method of treating and/or preventing chronic obstructive pulmonary disease (COPD) in a subject, the method comprising administering to the subject an effective amount of a combination of L-arginine and L-citrulline.

1. A nutritional composition, supplement, or fortifier comprising L-arginine, L-citrulline, a vitamin mixture, and a mineral mixture.

2. The nutritional composition, supplement, or fortifier according to claim 1, wherein the L-arginine is present in an amount such that the nutritional composition, supplement, or fortifier provides L-arginine in a dosage of about 0.5 g/day or more and L-citrulline in a dosage of about 0.5 g/day or more.

3. The nutritional composition, supplement, or fortifier according to claim 1, wherein the L-arginine and the L-citrulline are present in a weight ratio from about 0.1:1 to about 10:1.

4. The nutritional composition, supplement, or fortifier according to claim 1, wherein the vitamin mixture comprises vitamin A, vitamin D2, vitamin E, vitamin C, vitamin B1, vitamin B2, niacin, vitamin B6, folic acid, and vitamin B12.

5. The nutritional composition, supplement, or fortifier according to claim 1, wherein the mineral mixture comprises potassium, magnesium, iron, zinc, and selenium.

6. The nutritional composition, supplement, or fortifier according to claim 1, wherein the nutritional composition, supplement, or fortifier is a food for special dietary uses.

7. The nutritional composition, supplement, or fortifier according to claim 1, wherein the nutritional composition, supplement, or fortifier is a nutritional supplement.

8. A method of dietary management of a subject with an infection and/or having an immunodeficiency; enhancing an immune response; treating, preventing, and/or reducing a risk of occurrence or re-occurrence of an infection; adjunctive therapy in combination with an antimicrobial therapy; treating, preventing, and/or reducing a risk of occurrence of one or more symptoms of infection, wherein the symptoms of infection are selected from the group consisting of (i) positive sputum culture, (ii) cough, (iii) chest pain, (iv) fever, (v) night sweating, (vi) haemoptysis, (vii) dyspnea and (viii) GI symptoms of diarrhea or constipation; and/or treating, preventing, and/or reducing a risk of occurrence or reoccurrence of tuberculosis (TB) in a subject, the method comprising administering a nutritional composition, supplement, or fortifier to the subject, wherein the nutritional composition, supplement, or fortifier comprises L-arginine, L-citrulline, a vitamin mixture, and a mineral mixture.

9-13. (canceled)

14. A method of enhancing an immune response in a subject, the method comprising administering a combination of L-arginine and L-citrulline to the subject.

15. A method of treating, preventing, and/or reducing a risk of occurrence or reoccurrence of an infection in a subject, the method comprising administering a combination of L-arginine and L-citrulline to the subject.

16. The method of claim 15, wherein the infection is tuberculosis (TB).

17. The nutritional composition, supplement, or fortifier according to claim 1, wherein the L-arginine and the L-citrulline are present in a weight ratio of about 1:1.

18. The nutritional composition, supplement, or fortifier according to claim 6, wherein the food for special dietary uses is a powder for reconstitution with water or milk.

19. The nutritional composition, supplement, or fortifier according to claim 6, wherein the food for special dietary uses is nutritionally complete.

20. The nutritional composition, supplement, or fortifier according to claim 7, wherein the nutritional supplement is a powder supplement for sprinkling on food or in a beverage.
